(19) United States
(12) Patent Application Publication
(10) Pub. No.: US 2023/0174662 A1
(43) Pub. Date: Jun. 8, 2023

(54) METHODS OF TREATING CASTRATE-RESISTANT PROSTATE CANCER

(71) Applicant: FONDAZIONE PER L'ISTITUTO ONCOLOGICO DI RICERCA (IOR), Bellinzona (CH)

(72) Inventors: Abdullah AL AJATI, Bellinzona (CH); Mariantonietta D'AMBROSIO, Bellinzona (CH); Andrea ALIMONTI, Bellinzona (CH)

(21) Appl. No.: 17/426,760

(22) PCT Filed: Feb. 3, 2020
(86) PCT No.: PCT/EP2020/052626

§ 371 Date: Jul. 29, 2021

(30) Foreign Application Priority Data
Feb. 1, 2019 (EP) 19155128.2

(51) Int. Cl. C07K 16/28; A61K 39/395; A61P 35/00; A61P 13/08; A61K 31/4166; A61K 45/06; A61K 47/69; A61K 31/704; A61K 47/68; G01N 33/574

(52) U.S. Cl. CPC C07K 16/2896; A61K 39/39566; A61P 35/00; A61P 13/08; A61K 31/4166; A61K 45/06; A61K 47/6913; A61K 31/704; A61K 47/6869; A61K 47/6803; G01N 33/57434

(57) ABSTRACT

The invention relates to a downmodulator of CUB domain-containing protein 1 (CDCP1), for use in a method of treating a patient suffering from castrate-resistant prostate cancer. The invention further relates to a pharmaceutical composition, comprising a downmodulator of CDCP1 and a senolytic compound, and to methods of selecting a patient with prostate cancer eligible for treatment with a combination of downmodulator of CDCP1 and a senolytic compound.

The invention relates to methods for treatment of cancer by inducing senescence in cancer cells. Said cancer is prostate cancer, especially castrate-resistant prostate cancer.

### SEQUENCE LISTING

The instant application contains a Sequence Listing which has been filed electronically in ASCII format and is hereby incorporated by reference in its entirety. Said ASCII copy, created on Apr. 6, 2022 is named 14834-701-US0_SequenceListing.txt and is 48.3 KB in size.

### 1 INTRODUCTION

Castration-resistant prostate cancer (CRPC) is still the second leading cause of death between men in the Western Word. Although second generation androgen-deprivation therapies (ADT) have been successfully used to treat metastatic CRPC (mCRPC) patients, patients develop resistance and eventually succumb for this disease. Mechanisms of resistance in mCRPCs include Androgen Receptor (AR) activation (e.g. AR amplification, mutations or splicing variants) and the up regulation of signaling pathway that promote AR independent growth such the PI3K/AKT and MAPK pathways. Although in the majority of metastatic prostate cancers the PI3K signaling pathway is activated trough loss or mutations of PTEN, the mechanism by which the MAPK pathway becomes activated remains unknown. Indeed overexpression or mutations of both K-RAS and BRAF, two major regulators of this pathway accounts only for a minority of prostate cancer cases (El Sheikh et al., 2008. Neoplasia 10: 949-953; Reid et al., 2010. Br J Cancer 102: 678-684; Taylor et al., 2010. Cancer Cell 18: 11-22). Thus identification of new regulators of the MAPK pathway in the contest of PTEN null prostate cancer would open the way to new potentially effective therapies to treat these patients.

### 2 BRIEF DESCRIPTION OF THE INVENTION

The invention provides a downmodulator of CUB domain-containing protein 1 (CDCP1), for use in a method of treating a patient suffering from castrate-resistant prostate cancer.

The inventors surprisingly found that CDCP1 become upregulated in the course of treatment of prostate cancer patients, especially during treatment with anti-androgen therapy. A downmodulator of CDCP1 was found to induce senescence in these cells.

Said downmodulator preferably is or comprises an antibody that recognizes an extracellular epitope of CUB domain-containing protein 1 (CDCP1).

Said downmodulator for use according to the invention preferably is combined with anti-androgen therapy, preferably with an androgen receptor antagonist. Said anti-androgen preferably is selected from enzalutamide, abiraterone, bicalutamide, and nilutamide.

Said downmodulator for use according to the invention preferably is combined with a senolytic compound and/or a genotoxic agent, preferably in further combination with an androgen receptor antagonist such as enzalutamide, abiraterone, bicalutamide, and nilutamide.

Said senolytic compound preferably is selected from rapamycin, ABT263, FOXO4-DRI, a CXCR4 inhibitor and/or antagonist, and dasatinib.

The invention further provides a pharmaceutical composition, comprising a downmodulator of CDCP1 and an androgen receptor antagonist, a senolytic compound and/or a genotoxic agent. Said pharmaceutical composition preferably comprises a pharmaceutical preparation comprising the downmodulator of CDCP1, and a pharmaceutical preparation comprising the androgen receptor antagonist, the senolytic compound and/or the genotoxic agent.

The downmodulator of CDCP1 in a pharmaceutical composition according to the invention is preferably present in liposomes, said liposomes preferably further comprising an anthracycline such as doxorubicin.

A pharmaceutical preparation according to the invention preferably is for use in a method of treating a patient suffering from castrate-resistant prostate cancer.

The invention further provides a method of selecting a patient with prostate cancer eligible for treatment with a combination of downmodulator of CDCP1 and a senolytic compound, comprising determining if a level of testosterone in a bodily fluid of the patient; identifying a patient of which the level of testosterone is below 50 ng/dL; determining whether the prostate cancer is progressing in the identified patient; and selecting a patient in which the testosterone level is below 50 ng/dL and in which prostate cancer is progressing as a patient who is eligible for treatment with a combination of a downmodulator of CDCP1 and a senolytic compound.

Progression of prostate cancer in the identified patient is preferably determined by a continuous rise in serum prostate-specific antigen (PSA) levels, and/or the appearance of new metastases in said patient.

The invention further provides a method of treating a patient with prostate cancer with a combination of a downmodulator of CDCP1 and a senolytic compound, comprising determining a level of testosterone in a bodily fluid of the patient; identifying a patient of which the level of testosterone is below 50 ng/dL; determining whether the prostate cancer is progressing in the identified patient; and treating a patient in which the testosterone level is below 50 ng/dL and in which prostate cancer is progressing as a patient with a combination of a downmodulator of CDCP1 and a senolytic compound and/or genotoxic agent, preferably wherein the downmodulator is administered prior to the administration of the senolytic compound and/or genotoxic agent.

The invention further provides a method of treating a patient with castrate-resistant prostate cancer, comprising identifying a patient who suffers from castrate-resistant prostate cancer; and treating said identified patient with a combination of a downmodulator of CDCP1 and a senolytic compound and/or genotoxic agent, preferably wherein the downmodulator is administered prior to the administration of the senolytic compound and/or genotoxic agent.

### 3 FIGURE LEGENDS

FIG. 1. Advanced prostate tumours exhibit elevated expression of CUB domain-containing protein 1 (CDCP1) and conditional overexpression of CDCP1 promotes prostate tumourigenesis in transgenic mouse model. A. Representative images of IHC staining of CDCP1 in normal prostate, advanced/metastatic PCa and distant metastases in TMA of human prostate cancers. B. Percentage of CDCP1 positive samples in normal prostate/benign, localized HSPC, primary CRPC, advanced/metastatic PCa and distant metastases in TMA (n=438) of human prostate cancers. C. Histopathological characterization and quantification of the prostate in WT and CDCP1 mice. (BPH: Benign prostatic hyperplasia). D. Quantification of Ki-67 staining in anterior prostate of WT and CDCP1 mice at the indicated ages (n=3 for each genotype). E. Bar graph represents the fold change of normalized p-Akt, p-Erk1/2 and p-Src to their total proteins in CDCP1 prostates compared to WT prostates (n=4). Error bars indicate standard deviation (SD). *P<0.05; **P<0.01. n.s, non-significant.

FIG. 2. CDCP1 cooperates with Pten-loss in driving full malignancy and metastasis in prostate cancer. A. Representative images of H&E-staining of anterior prostate in Ptenpc^{-/-} and CDCP1; Ptenpc^{-/-} at the age of 10 months. Original magnification, 10×. Insets are regions shown in higher magnification (40×) of each genotype. B. Quantification of Ki-67 staining in anterior prostate of indicated genotypes (n=4 for each genotype). Error bars indicate SD. *P<0.05. C. Cumulative survival of WT, CDCP1; Ptenpc^{-/-} and CDCP1; Ptenpc^{-/-} mice. Insets represent anterior prostate of Ptenpc^{-/-} and CDCP1; Ptenpc−/−. Scale of lcm. D. Western blot analysis and protein fold change quantification of specified proteins in prostate anterior glands from the indicated genotypes at 20 weeks of age. E. Quantification of anterior prostate weights and volume of Ptenpc^{-/-} and CDCP1; Ptenpc^{-/-} castrated and non-castrated prostates at 8 weeks post-castration mice (n=4). F. Western blot analysis and protein fold change quantification of indicated protein in the anterior prostate of Ptenpc−/− and CDCP1; Ptenpc−/− castrated mice at 20 weeks of age. Error bars indicate SD. n.s, non-significant; *P<0.05; **P<0.01; ***P<0.001.

FIG. 3. Overexpression of CDCP1 overcomes Pten-loss induced cellular senescence by activating c-Myc. A. Western blot analysis of p21, c-Myc, Cyclin D1, COUP-TFII, Smad4 and p53 in prostate anterior glands from the indicated genotypes. B. Quantitative real-time PCR analysis of c-Myc, Cyclin D, COUP-TF-II, p21, p27, and p16 expression in Ptenpc^{-/-} and CDCP1; Ptenpc^{-/-} prostates from 12-16 weeks old mice (n=3 mice). C. Western blot analysis of Pten^{-/-} and CDCP1; Pten^{-/-} MEFs treated with saracatinib (100 nM) for 12 hr. D. Quantification of fold change in growth by crystal violet in Pten^{-/-} and CDCP1; Pten^{-/-} MEFs treated with saracatinib (100 nM) or DMSO as control (n=3).

FIG. 4. A. Western blot analysis of the CDCP1 protein in infected PC3 cells expressing PLKOsh-CDCP1 (sh-CDCP1 #1) and doxycycline inducible Tripz-sh-CDCP1 (sh-CDCP1 #2). B. Bar graph represents percentage of SA-β-Gal positive cells in all groups (n=3).

FIG. 5. Left panel, Western blot analysis of CDCP1, p-SRC, SRC, c-MYC, CYCLIN D1, COUP-TFII in PC3 sh-Cont #2 and PC3 sh-CDCP1 #2 xenografts tumour. Right Panel, Quantitative real time PCR of p27 and p21 mRNA levels in PC3 sh-Cont #2 and PC3 sh-CDCP1 #2 xenografts tumour (n=3).

FIG. 6. Advanced prostate tumours exhibit elevated expression of CDCP1 A. Expression (H-score) of membranous CDCP1 in matched biopsies at HSPC and CRPC stage in 26 prostate cancer patients. Median H-scores and interquartile range are shown. p-values were calculated using the Wilcoxon matched-pair signed rank test. B. Left panel, Western blot analysis of CDCP1, p-SRC, SRC, c-MYC, p-ERK1/2 and ERK1/2 in LNCaP-ADS and LNCaP-ADI. Right panel, Quantification of fold change in CDCP1 protein levels in LNCaP-ADS and LNCaP-ADI. C. Left panel, Quantitative real time PCR analysis of CDCP1 mRNA levels in LNCaP grown in full media, full androgen depreviation (FAD) and stimulated with dihydrotestosterone (DHT 1 μM, 16 h) after grown for 2 days in FAD. Right panel, Western blot analysis of CDCP1, p-SRC, SRC, p-AKT, AKT, p-ERK1/2 and ERK1/2 in LNCaP grown in full media, FAD and stimulated with DHT (1 μM, 16 h) after grown for 2 days in FAD. D. Left panel, Quantitative real time PCR of CDCP1 mRNA levels in PC3 expressing empty vector (PC3-Cont) and in PC3 overexpressing full-length Androgen Receptor (PC3-AR). Middle panel, Quantification of fold change in CDCP1 protein levels in PC3-cont and PC3-AR. Right panel, Western blot analysis of CDCP1 and AR in PC3-Cont and PC3-AR cell lines.

FIG. 7A. Left panel, Quantification of fold change in growth by crystal violet in LNCaP kept in full media and in LNCaP kept in full media and treated with mAb-CUB4. Right panel, Quantification of fold change in growth by crystal violet in LNCaP kept in FAD and in LNCaP kept in FAD and treated with mAb-CUB4. B. Left panel, Xenografts tumour growth (mm3) of LNCaP cell line untreated, treated with Enzalutamide, Immuno-liposome and treated with the combination of Enzalutamide and immuno-liposome. Right panel, quantification of fold change in protein levels of Cleaved Caspase 3 in LNCaP xenografts groups. C. Western blot analysis of CDCP1 expression, p-SRC and Cleaved Caspase 3 in LNCaP xenografts groups.

FIG. 8. Anti-CDCP1 antibody sequences. A. Mouse and chimeric mouse-human heavy chain sequences, as depicted in WO2015/082446. B. Human heavy chain sequences, as depicted in WO2011/023389 and in Siva et al., 2008 (Siva et al., 2008. J Immunol Methods 330: 109-119). C. Mouse and chimeric mouse-human light chain sequences, as depicted in WO2015/082446. D. Human lighy chain sequences, as depicted in WO2011/023389 and in Siva et al., 2008 (Siva et al., 2008. J Immunol Methods 330: 109-119).

FIG. 9. Association of PTEN genomic loss to CDCP1 gene expression in the Cancer Genome Atlas (TCGA; left panel) and Taylor dataset (right panel; Taylor et al., 2010. Cancer Cell 18: 11-22). Error bars indicate standard errors of the mean (SEM) Statistical test: Kruskal-Wallis (Kruskal and Wallis, 1952. J American Statistical Association 47: 583-621).

FIG. 10. Association of PTEN and CDCP1 expression levels with disease-free survival in the indicated patient datasets. In Taylor dataset, low PTEN indicates patients with expression signal lower than 8.74, and high CDCP1 indicates patients with expression signal higher than 11.19. In TCGA, low PTEN indicates patients with expression signal lower than 10.19, and high CDCP1 indicates patients with expression signal higher than 9.49. HR, hazard ratio. Statistical test: Mantel-Cox test (Mantel, 1966. Cancer Chemotherapy Reports 50: 163-170).

### 4 DETAILED DESCRIPTION OF THE INVENTION

### 4.1 Definitions

The term “downmodulator”, as is used herein, refers to a molecule that reduces expression of CUB domain-containing protein 1 (CDCP1), especially the expression of CDCP1 on the surface of castrate-resistant prostate cancer cells, and/or a molecule that reduces CDCP1-mediated MAPK activation in castrate-resistant prostate cancer cells.

The term “CUB domain”, as is used herein, refers to a structural motif of approximately 110 amino acid residues that was firstly identified in the proteins Complement component 1r/1s, sea urchin protein epidermal growth factor (uEGF) and Bone Morphogenetic protein 1. The CUB domain is an evolutionarily conserved protein domain that is almost exclusively present in extracellular and plasma membrane-associated proteins.

The term “CUB domain-containing protein 1 (CDCP1)”, as is used herein, refers to a protein product of a CDCP1 gene located on human chromosome 3p21.31 The CDCP1 gene encodes a transmembrane protein which contains three extracellular CUB domains and acts as a substrate for Src family kinases. The protein plays a role in the tyrosine phosphorylation-dependent regulation of cellular events that are involved in tumor invasion and metastasis. Alternative splicing results in multiple transcript variants of this gene. The gene is referred to under HGNC: 24357; Entrez Gene: 64866; and/or Ensembl: ENSG00000163814. The human protein is referred to under UniProt: Q9H5V8. CDCP1 comprises a total of 836 amino acid residues, of which the N-terminal amino acid residues 1-29 represent a signal peptide; amino acid residues 30-668 is an extracellular part and comprises the 3 CUB domains; a region between amino acid residues 668 and 688 represents a membrane-spanning region; and the C-terminal region from amino acid residue 689 represents a cytoplasmic domain.

The term “extracellular epitope of CDCP1”, as is used herein, refers to one or more epitopes in the N-terminal extracellular part of CDCP1, comprising amino acid residues 30-668.

The term “castrate-resistant prostate cancer”, as is used herein, refers to prostate cancer cells that continue to proliferate when androgen in the body is reduced or even absent. Many early-stage prostate cancers depend on androgen in order to proliferate, but castrate-resistant prostate cancers do not. Also called CRPC.

The term “androgen”, as is used herein, refers to a natural or synthetic steroid hormone that binds to androgen receptors. Androgens are synthesized in the testes, the ovaries, and the adrenal glands. A major androgen is testosterone, but also dihydrotestosterone and androstenedione are included under the term androgen.

The term “anti-androgen therapy”, as is used herein, refers to a therapy that is aimed to reduce levels of androgens in the body, or to stop them from affecting prostate cancer cells. Alternative terms include androgen deprivation therapy (ADT) and androgen suppression therapy. Said therapy includes the use of luteinizing hormone-releasing hormone (LHRH) agonists (also called LHRH analogs or GnRH agonists) and LHRH antagonist such as leuprolide (L-pyroglutamyl-L-histidyl-L-tryptophyl-L-seryl-L-tyrosyl-D-leucyl-L-leucyl-L-arginyl-L-proline ethylamide acetic acid), goserelin (L-pyroglutamyl-L-histidyl-L-tryptophyl-L-seryl-L-tyrosyl-O-tert-butyl-D-seryl-L-leucyl-L-arginyl-N′-carbamoyl-L-prolinehydrazide), triptorelin (L-pyroglutamyl-L-histidyl-L-tryptophyl-L-seryl-L-tyrosyl-D-tryptophyl-L-leucyl-L-arginyl-L-prolyl-glycinamide), histrelin (L-pyroglutamyl-L-histidyl-L-tryptophyl-L-seryl-L-tyrosyl-1-benzyl-D-histidyl-L-leucyl-L-arginyl-L-proline ethylamide), and degarelix (N-acetyl-3-(2-naphthyl)-D-alanyl-4-chloro-D-phenylalanyl-3-(3-pyridyl)-D-alanyl-L-seryl-4-((S)-dihydroorotamido)-L-phenylalanyl-4-ureido-D-phenylalanyl-L-leucyl-N6-isopropyl-L-lysyl-L-prolyl-D-alaninamide); a CIP17 inhibitor and/or androgen synthesis inhibitor such as abiraterone ((3S,8R,9S,10R,13S,14S)-10,13-dimethyl-17-pyridin-3-yl-2,3,4,7,8,9,11,12,14,15-decahydro-1H-cyclopenta[a]phenanthren-3-01); an androgen synthesis inhibitor such as ketoconazole (1-[4-[4-[[(2R,4S)-2-(2,4-dichlorophenyl)-2-(imidazol-1-ylmethyl)-1,3-dioxolan-4-yl]methoxy]phenyl]piperazin-1-yl]ethanone), TAK-700 (orteronel; 6-[(7S)-7-hydroxy-5,6-dihydropyrrolo[1,2-c]imidazol-7-yl]-N-methylnaphthalene-2-carboxamide), and TOK-001 (galeterone; (3S,8R,9S,10R,13S,14S)-17-(benzimidazol-1-yl)-10,13-dimethyl-2,3,4,7,8,9,11,12,14,15-decahydro-1H-cyclopenta[a]phenanthren-3-01), and an androgen receptor antagonist such as flutamide (2-methyl-N-[4-nitro-3-(trifluoromethyl)phenyl] propanamide), bicalutamide (N-[4-cyano-3-(trifluoromethyl)phenyl]-3-(4-fluorophenyl)sulfonyl-2-hydroxy-2-methylpropanamide), nilutamide (5,5-dimethyl-3-[4-nitro-3-(trifluoromethyl)phenyl]imidazolidine-2,4-dione), ARN-509 (apalutamide; 4-[7-[6-cyano-5-(trifluoromethyl)pyridin-3-yl]-8-oxo-6-sulfanylidene-5,7-diazaspiro[3.4]octan-5-yl]-2-fluoro-N-methylbenzamide), and enzalutamide (4-[3-[4-cyano-3-(trifluoromethyl)phenyl]-5,5-dimethyl-4-oxo-2-sulfanylideneimidazolidin-1-yl]-2-fluoro-N-methylbenzamide).

The term “genotoxic agent”, as is used herein, refers to an agent that induces damage in the genomic DNA of a cell, including base modifications, single strand breaks, and crosslinks, such as intrastrand and interstrand cross-links.

The term “senolytic compound”, as is used herein, refers to compounds that eliminate senescent cells, which cells has lost function, but are resistant to cell death. Said elimination preferably is selective, meaning that is the number of senescent cells is reduced, preferably to zero, while the number of non-senescent cells is not reduces, or only hardly reduced. Examples of senolytic compounds, also termed senolytics, are dasatinib, quercetin and a combination thereof (Xu et al., 2018. Nature Med 24:1246-1256), UBX0101 (also termed Navitoclax and ABT-263; Jeon et al., 2017. Nature Med 23: 775-781), piperlongumine and analogues thereof (Zhu et al., 2019. Bioorganic Med Chem 26: 3925-3938), fisetin, a naturally-occurring flavone (Zhu et al., 2917. Aging 9: 955-963); selective BCL-XL inhibitors such as A1331852 and A1155463 (Zhu et al., 2917. Aging 9: 955-963), HSP90 chaperone inhibitors such as 17-DMAG (Fuhrmann-Stroissnigg et al., 2017. Nature Comm 8: 422 (10.10381s41467-017-00314-z); acarbose, nordihydroguaiaretic acid (NDGA) (Harrison et al., 2014. Aging Cell 13: 273-282); and FOXO4-DRI (H-D-Leu-D-Thr-D-Leu-D-Arg-D-Lys-D-Glu-D-Pro-D-Ala-D-Ser-D-Glu-D-Ile-D-Ala-D-Gln-D-Ser-D-Ile-D-Leu-D-Glu-D-Ala-D-Tyr-D-Ser-D-Gln-D-Asn-D-Gly-D-Trp-D-Ala-D-Asn-D-Arg-D-Arg-D-Ser-D-Gly-D-Gly-D-Lys-D-Arg-D-Pro-D-Pro- D-Pro-D-Arg-D-Arg-D-Arg-D-Gln-D-Arg-D-Arg-D-Lys-D-Lys-D-Arg-D-Gly-OH; Baar et al., 2017. Cell 169: 132-147).

The term antibody, as is used herein, includes classical heterodimers of heavy and light chain antibodies, single heavy chain variable domain antibody such as a camelid VHH, a shark immunoglobulin-derived variable new antigen receptor, and scFv, a tandem scFv, a scFab, and an improved scFab (Koerber et al., 2015. J Mol Biol 427: 576-86). The term also includes alpha-beta T-cell receptor molecules and/or gamma-delta T cell receptor molecules that specifically bind CDCP1, both isolated T cell receptor molecules and T cell receptor molecules that are present on the surface of T cells, such as Chimere Antigen Receptor-T cells (CAR T cells).

The term antibody also includes a antibody-like molecule that can specifically bind CDCP1, but that is not structurally related to an antibody. Such antibody-like molecules include a designed ankyrin repeat protein, a binding protein that is based on a Z domain of protein A, a binding protein that is based on a fibronectin type III domain, engineered lipocalin, and a binding protein that is based on a human Fyn SH3 domain (Skerra, 2007. Current Opinion Biotechnol 18: 295-304; Škrlec et al., 2015. Trends Biotechnol 33: 408-418).

The term antibody also provides reference to an antibody-drug conjugate, whereby the drug conjugate is a chemotherapeutic drug, a toxic compound or a radioactive compound. Examples of a toxic compound are saporin, bryodin, agrostin, ricin, gelonin, dianthin, luffin, α-momorcharin, ß-momorcharin, dodecandrin, tritin, momordin, and trichosanthin.

The term ‘specific binding’ or ‘specificity’ or grammatical variations thereof refer to the number of different types of antigens or their epitopes to which a particular antibody can bind. The specificity of an antibody can be determined based on affinity. A specific antibody preferably has a binding affinity Kd for its epitope of less than 10⁻⁷ M, preferably less than 10⁻⁸ M, most preferable less than 10⁻⁹ M.

### 4.2 Upregulation of CDCP1

The invention provides a downmodulator of CUB domain-containing protein 1 (CDCP1), for use in a method of treating a patient suffering from castrate-resistant prostate cancer.

A prostate cancer has become castrate resistant if cancer progresses despite a plasma testosterone level between 20 and 50 ng/dL, which is equivalent between 0.7 and 1.7 nmol/L, after androgen deprivation therapy. Symptomatic progression alone must be questioned and subject to further investigation. Symptomatic progression by itself is not sufficient to diagnose CRPC. Further markers for castrate resistant prostate cancer (CRPC) include biochemical progression, for example by three consecutive rises in prostate-specific antigen (PSA) one week apart resulting in two 50% increases over the nadir, and/or a PSA>2 ng/mL, and/or a radiological progression, for example the appearance of a new lesion such as, for example, one, two or more new bone lesions on a bone scan or a soft tissue lesion using RECIST (Response Evaluation Criteria in Solid Tumours) (EAU Guidelines for Prostate Cancer, 2017. N. Mottet).

It was found in the present invention that CDCP1 becomes upregulated in prostate cancer cells upon treatment with anti-androgen therapy, especially upon treatment with an androgen receptor antagonist. Upregulation becomes prominent at the time that prostate cancer cells become resistant to said anti-androgen therapy. Without being bound by theory, it is thought that upregulation of CDCP1 provides an independent driving force that stimulates the MAPK-pathway to such extent that the cells become independent of androgens, i.e., resistant to anti-androgen therapy. Downmodulation of the expression of CDCP1 and/or of the MAPK-pathway stimulating activity of CDCP1, will induce senescence in such castrate resistant prostate cancer cells.

Said downmodulator is a molecule that downmodulates expression of CDCP1 in prostate cancer cells and/or a molecule that inactivates CDCP1-mediated MAPK activation in prostate cancer cells.

### 4.3 Molecules that Downmodulate Expression of CDCP1 in Prostate Cancer Cells

A molecule that downmodulates expression of CUB domain-containing protein 1 (CDCP1) on the surface of prostate cancer cells preferably is selected from a molecule that downmodulates or abolishes transcription of the CDCP1 gene, such as a zinc-finger protein transcription factor, a transcription activator-like effector (TALE) repressor, and disruption of the CDCP1 gene, for example mediated by a transcription activator-like effector nuclease (TALEN) or by Clustered Regularly Interspaced Short Palindromic Repeats (CRISPR) and CRISPR-associated protein CAS (Gaj et al., 2013. Trends Biotechnol 31: 397-405); downmodulation of RNA expression products by, for example an antisense RNA molecule and a siRNA molecule; and downmodulation of the CDCP1 protein by, for example, an antibody.

Zinc finger protein transcription factors are DNA-binding motifs and consist of modular zinc finger domains that are coupled to a transcriptional activator or repressor. Each domain can be engineered to recognize a specific DNA triplet. A combination of three or more domains results in the recognition of a gene-specific sequence. Said assembled zinc finger protein is coupled to a transcriptional repressor domain, for example a Kruppel-associated box (KRAB), an ERF repressor domain (ERD), or a mSIN3 interaction domain (SID). Expressing said coupled zinc finger protein-transcriptional repressor domain in a prostate cancer cell will result in silencing of the CDCp1 gene.

Similarly, synthetic transcription factor DNA binding domains (DBDs) can be programmed to recognize specific DNA motifs. Such transcription activator-like effector (TALE) DNA binding domains (DBD) contain a number, from 7 to 34, highly homologous direct repeats, each consisting of 33-35 amino acids. Specificity is contained in the two amino acid residues in positions 12 and 13 of each repeat. Since the DNA:protein binding code of RVDs has been deciphered, it is possible to design TALEs that bind any desired target DNA sequence by engineering an appropriate DBD. Typically, the TALEs are designed to recognize 15 to 20 DNA base-pairs, balancing specificity with potential off targeting (Boettcher and McManus, 2015. Mol Cell 58: 575-585). A CDCP1-specific TALE is than coupled to a transcriptional repressor domain, for example a KRAB domain, an ERD domain, or a SID domain. Expressing said coupled TALE-transcriptional repressor domain in a prostate cancer cell will result in silencing of the CDCp1 gene.

TALEN or CRISPR-CAS mediated disruption of the CDCP1 gene is mediated by targeting a nuclease to at least one specific position on the CDCP1 gene, preferably at least two specific positions. Said targeting is mediated by the TALE-DNA binding domains, or by the CRISPR single chimeric guide RNA sequences. The nuclease, a FOK1 nuclease in the case of a TALEN, and a CAS protein, preferably a CAS9 protein, for CRISPR, mediates double stranded breaks in the genomic DNA of the CDCP1 gene. The introduction of DNA double stranded breaks increases the efficiency of gene editing via homologous recombination, in the presence of suitable donor DNA to delete a part or all of theCDCP1 gene (Gaj et al., 2013. Trends Biotechnol 31: 397-405).

Antisense RNA, or antisense oligonucleotide, employs one or more single stranded RNA molecules that are complementary to a CDCP1 protein coding messenger RNA (mRNA). Said antisense RNA will hybridize to the mRNA and thereby blocks its translation into protein. As RNA molecules are easily degraded by RNase or other degrading enzymes, chemical modification is usually required. The most common chemical modification on the oligonucleotides is adding a phosphorothioate linkage to the backbone.

A further preferred antisense RNA molecule comprises a modified backbone, such as a morpholino backbone, carbamate backbone, siloxane backbone, sulfide, sulfoxide and sulfone backbone, formacetyl and thioformacetyl backbone, methyleneformacetyl backbone, riboacetyl backbone, alkene containing backbone, sulfamate, sulfonate and sulfonamide backbone, methyleneimino and methylenehydrazino backbone, and amide backbone. Morpholino antisense RNA molecules have an uncharged backbone in which the deoxyribose sugar of DNA is replaced by a six membered ring and the phosphodiester linkage is replaced by a phosphorodiamidate linkage. Morpholino oligonucleotides are resistant to enzymatic degradation.

In a further preferred antisense RNA molecule, the linkage between the ribonucleotide residues in a backbone do not include a phosphorus atom, such as a linkage that is formed by short chain alkyl or cycloalkyl-internucleoside linkages, mixed heteroatom and alkyl or cycloalkyl-internucleoside linkages, or one or more short chain heteroatomic or heterocyclic internucleoside linkages.

A preferred antisense RNA molecule comprises a substitution of one of the non-bridging oxygens in the phosphodiester linkage. This modification slightly destabilizes base-pairing but adds significant resistance to nuclease degradation. A preferred nucleotide analogue or equivalent comprises phosphorothioate, chiral phosphorothioate, phosphorodithioate, phosphotriester, aminoalkylphosphotriester, H-phosphonate, methyl and other alkyl phosphonate including 3′-alkylene phosphonate, 5′-alkylene phosphonate and chiral phosphonate, phosphinate, phosphoramidate including 3′-amino phosphoramidate and aminoalkylphosphoramidate, thionophosphoramidate, thionoalkylphosphonate, thionoalkylphosphotriester, selenophosphate or boranophosphate. These substitutions render the antisense RNA molecule RNase H and nuclease resistant and increase the affinity for the target RNA.

A further preferred antisense RNA molecule comprises a Peptide Nucleic Acid (PNA), having a modified polyamide backbone (Nielsen et al., 1991. Science 254: 1497-1500), or comprises a Locked Nucleic Acid (LNA), in which the 2′-carbon atom is linked to the 3′ or 4′ carbon atom of the sugar ring thereby forming a bicyclic sugar moiety. A preferred LNA comprises 2′-O,4′-C-ethylene-bridged nucleic acid (Morita et al. 2001. Nucleic Acid Res Supplement No. 1: 241-242).

RNA interference (RNAi) i is based on the generation of short, double-stranded RNA (dsRNA) which activates a cellular process leading to a highly specific RNA degradation (Zamore et al., 2000. Cell 101: 25-33) and/or suppression of translation. For the purpose of the invention, the dsRNA molecules that activate RNAi and their precursors that are processed in a cell to generate dsRNA molecules that activate RNAi are referred to as “RNAi molecules”. RNA interference is mediated by the generation of 18- to 23-nucleotide dsRNA molecules with 2 nucleotide-long 3′ overhangs termed small interfering RNA (siRNA) duplexes. RNAi allows silencing of a gene on the basis of its sequence. Preferably, an RNAi molecule is a molecule that can activate an RNAi process in a cell either directly or indirectly because it is a precursor of a molecule that can activate an RNAi process in a cell. Said precursor molecule is preferably an shRNA or a pre- or pri-miRNA or variants or analogues thereof.

Said RNAi molecule, for example, is a short hairpin RNA (shRNA) or a miRNA precursor. A short hairpin RNA (shRNA) typically comprises a 50-100 nucleotide long RNA molecule comprising two stretches of nucleotides that are complementary and can base-pair, whereby the two stretches are interconnected through a hairpin turn. The shRNA hairpin structure is cleaved by the cellular machinery into 18-23 (typically 19) nucleotide-long double stranded siRNA molecules with 2 nucleotide-long 3′ overhangs with one of the strands exhibiting extensive complementary homology to a part of a mRNA transcript from a target gene. Said siRNA activates the RNA interference (RNAi) pathway and interferes with the expression of said target gene by specific mRNA degradation. Expression of the shRNA can be driven by a polymerase II or polymerase III enhancer/promoter. Natural miRNA molecules are typically transcribed by polymerase II as pri-miRNA with a cap and poly-A tail and processed to short, 70-nucleotide stem-loop structures known as pre-miRNA in the cell nucleus. These pre-miRNAs are then processed to mature double stranded miRNAs of about 18-25 nucleotides in the cytoplasm which silence gene expression via RNA interference, partly by specific RNA degradation and partly by suppressing translation. Pri-miRNAs and pre-miRNA molecules are also useful silencing factors according to the invention. Artificial miRNAs can be transcribed from any promoter, for example a polIII promoter, in a format analogous to that of a shRNA. They then differ from a shRNA in that the double-stranded region is not completely complementary.

A preferred RNAi molecule according to the invention comprises a double stranded region of between 18 nucleotides and 25 nucleotides per strand, such as 18 nucleotides, 19 nucleotides, 20 nucleotides, 21 nucleotides. 22 nucleotides, 23 nucleotides, 24 nucleotides, or 25 nucleotides. A most preferred RNAi molecule according to the invention comprises a double stranded region that has a length of 19 nucleotides after processing into a mature siRNA. Said preferred RNAi molecule comprises the sequences V3THS_329377: 5′-TGAGGGTAGGCAACAACGA and/or V2THS_191307: 5′-TCTTTCTCCAGACTTGATG.

A preferred method for downmodulation of RNA expression products comprises a heterogeneous mixture of antisense RNA molecules and/or a mixture of siRNAs that all target an CDCP1 mRNA sequence. Such multiple silencing lead to highly specific and effective gene silencing.

Said molecule that downmodulates expression of CUB domain-containing protein 1 (CDCP1) on the surface of prostate cancer cells preferably is provided in a vector. Said vector preferably additionally comprises means for high expression levels such as strong promoters, for example of viral origin (e.g., human cytomegalovirus) or promoters derived from genes that are highly expressed in a prostate cancer cell such as a PSA-, probasin-, or MMTV LTR-promoter (Lu et al., 2013. Biomed Res Int 624632). The vectors preferably comprise selection systems such as, for example, expression of glutamine synthetase or expression of dihydrofolate reductase for amplification of the vector in a suitable recipient cell, as is known to the skilled person.

Said vector preferably is a viral vector, preferably a viral vector that is able to transduce prostate cancer cells. Said viral vector preferably is a recombinant adeno-associated viral vector, a herpes simplex virus-based vector, or a retrovial vector such as a lentivirus-based vector, for example a human immunodeficiency virus-based vector. Said viral vector most preferably is a retroviral-based vector such as a lentivirus-based vector such as a human immunodeficiency virus-based vector, or a gamma-retrovirus-based vector such as a vector based on Moloney Murine Leukemia Virus (MoMLV), Spleen-Focus Forming Virus (SFFV), Myeloproliferative Sarcoma Virus (MPSV) or on Murine Stem Cell Virus (MSCV). A preferred retroviral vector is the SFG gamma retroviral vector (Rivière et al., 1995. PNAS 92: 6733-6737).

Retroviruses, including a gamma-retrovirus-based vector, can be packaged in a suitable complementing cell that provides Group Antigens polyprotein (Gag)-Polymerase (Pol) and/or Envelop (Env) proteins. Suitable packaging cells are human embryonic kidney derived 293T cells, Phoenix cells (Swift et al., 2001. Curr Protoc Immunol, Chapter 10: Unit 10 17C), PG13 cells (Loew et al., 2010. Gene Therapy 17: 272-280) and Flp293A cells (Schucht et al., 2006. Mol Ther 14: 285-92).

As an alternative, non-viral gene therapy may be used for transducing the molecule that downmodulates expression of CDCP1 on the surface of prostate cancer cells. Non-viral vectors include nude DNA, liposomes, polymerizers and molecular conjugates. Minicircle DNA vectors free of plasmid bacterial DNA sequences may be generated in bacteria and may express a nucleic acid encoding a molecule that downmodulates expression of CDCP1 in prostate cancer cells.

Preferred non-viral gene therapy comprises dual stealth immunoliposome carrying a downmodulator of CDCP1, preferably an anti-CDCP1 antibody. Said immunoliposome preferably further comprises a genotoxic agent such as an alkylating agent such as nitrogen mustard, e.g. cyclophosphamide, mechlorethamine or mustine, uramustine and/or uracil mustard, melphalan, chlorambucil, ifosfamide; nitrosourea, including carmustine, lomustine, streptozocin; an alkyl sulfonate such as busulfan, an ethylenime such as thiotepa and analogues thereof, a hydrazine/triazine such as dacarbazine, altretamine, mitozolomide, temozolomide, altretamine, procarbazine, dacarbazine and temozolomide; an intercalating agent such as a platinum-based compound like cisplatin, carboplatin, nedaplatin, oxaliplatin and satraplatin; anthracyclines such as doxorubicin, daunorubicin, epirubicin and idarubicin; mitomycin-C, dactinomycin, bleomycin, adriamycin, and mithramycin.

Said immunoliposomes are preferably prepared using a choline, preferably a phosphatidylcholine such as hydrogenated soy phosphatidylcholine, more preferably a combination of a choline and cholesterol, more preferably a combination of choline, cholesterol, and a phospholipid such as distearoylphophatidylethanolamine conjugated with poly-(ethylene glycol) (PEG), as is known to a person skilled in the art (Immordino et al., 2006. Int J Nanomedicine 1: 297-315). For incorporation into immunoliposomes, an anti-CDCP1 antibody preferably is coupled to PEG, more preferably to a PEG-phospholipid derivative such as, for example, distearoylphophatidylethanolamine conjugated with PEG.

A preferred molecule that downmodulates expression of CDCP1 on the cell surface of prostate cancer cells is an antibody, preferably an antibody that recognizes an extracellular epitope of CUB domain-containing protein 1 (CDCP1). Preferred antibodies recognize an extracellular epitope of CDCP1 and result in downmodulation of CDCP1 on the cell surface. induce senescence.

Suitable antibodies that specifically bind to the extracellular domain of CDCP1 are known in the art. For example, Bühring et al., 2004 (Bühring et al., 2004. Stem Cells 22: 334-343) describe mouse monoclonal antibodies against the extracellular domain of CDCP1. Further suitable antibodies have been described in Siva et al., 2008 (Siva et al., 2008. J Immunol Methods 330: 109-119) and in the published international patent applications WO2011/023389, WO2015/082446.

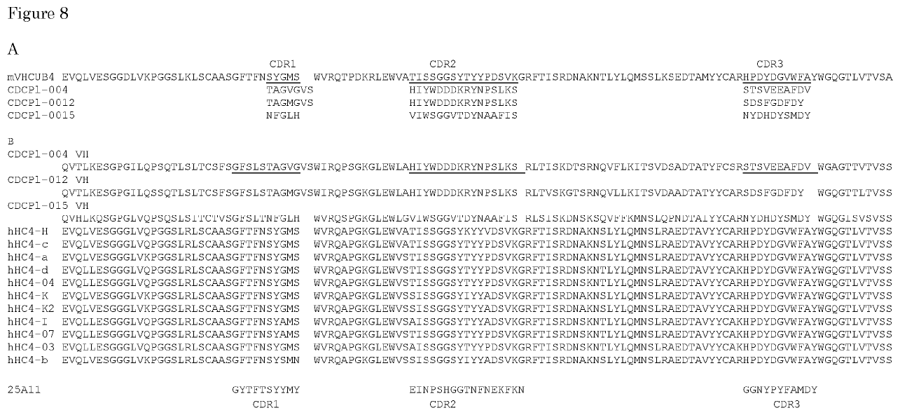

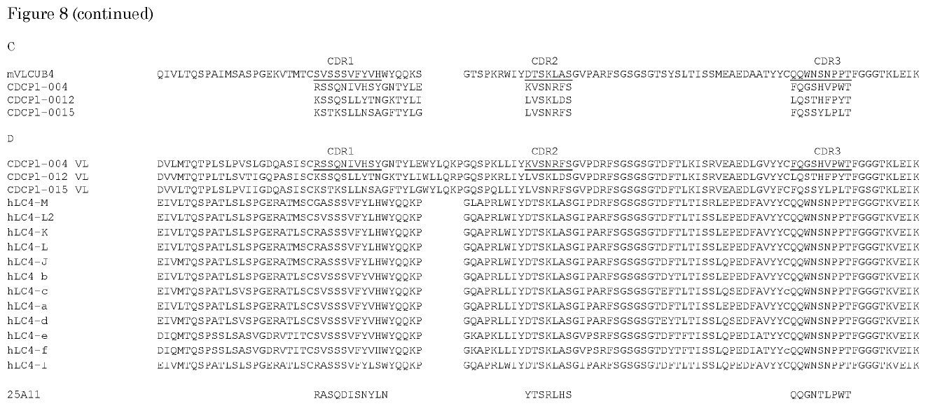

Said antibodies preferably comprise CDR1, CDR2 and CDR3 amino acid sequences as depicted in FIG. 8, more preferably the variable domain amino acid sequences as depicted in FIG. 8.

### 4.4 Molecules that Inactivate CDCP1-Mediated MAPK Activation in Prostate Cancer Cells

A molecule that downmodulates the MAPK-pathway stimulating activity of CDCP1, preferably is a small compound molecule. Said molecule preferably inhibits phosphorylation of CDCP1 by Src kinase, and/or inhibits the interaction between the Protein Kinace C-delta (PKCd) and phosphorylated CDCP1, more preferably between the C2 domain of PKCd and phosphorylated CDCP1.

Suitable molecules have been describes, for example by Nakashima et al., 2017 (Nakashima et al., 2017. Cancer Sci 108: 1049-1057). Said molecules preferably comprise a glycoconjugated palladium complex (Pd-Oqn). Preferred molecules include chloror{N-(hydroxo-quinoline-2-ylmethylidene)-b-D-glucosamine} palladium(II)} and chloror{N-(hydroxo-quinoline-2-ylmethylidene)-b-D-glucosamine} platinum(II), most preferably chloror{N-(hydroxo-quinoline-2-ylmethylidene)-b-D-glucosamine} palladium(II)}.

### 4.5 Methods of Treatment

The invention provides a downmodulator of CDCP1 for use in a method of treating a patient suffering from castrate-resistant prostate cancer.

Said downmodulator of CDCP1 is a molecule that downmodulates expression of CUB domain-containing protein 1 (CDCP1) on the surface of prostate cancer cells, or a molecule that downmodulates the MAPK-pathway stimulating activity of CDCP1 may be administered systemically. Systemic administration includes oral administration, intravenous, intramuscular, intra-articular, intra-arterial, intramedullary, intrathecal, epidural, intraventricular, transdermal, subcutaneous, intraperitoneal, intranasal, enteral, topical, sublingual, inhalational, intraocular, intra-aural or rectal injection or infusion, preferably intravenous or intramuscular infection or infusion.

The amount of a downmodulator of CDCP1 that is administered will be determined by the individual to which the dose is administered, in light of factors related to the individual's requiring treatment. Said dosage preferably is between 0.5 microgram and 10 milligram per kg per administration. Dosage and administration are adjusted to provide sufficient levels of the active agent or to maintain the desired therapeutic effect. Factors that can be taken into account include the severity of the prostate cancer and other factors, including the general health of the subject, age, weight and gender of the subject, diet, time and frequency of administration, drug combination(s), reaction sensitivities and tolerance/response to therapy, as is known to a person skilled in the art.

Suitable dosages of said inhibitors are 1-50 mg/kg of body weight, preferably about 10 mg/kg of body weight for an antisense RNA molecule and a siRNA molecule; 0.1-50 mg/kg of body weight, preferably about 0.5-2.5 mg/kg of body weight, for antibodies.

The downmodulator of CDCP1 may be administered once, twice or three times per day, once every other day, and/or once per week. A treatment regimen may last between 1-13 weeks, and may be interrupted by a period of 1-4 weeks of without administration. A treatment regimen may be repeated, if necessary, until a reduction of the prostate cancer load is obtained.

A downmodulator of CDCP1 for use according to the invention may be combined with anti-androgen therapy, a senolytic compound and/or a genotoxic agent.

The invention therefore provides a method of treating a patient suffering from prostate cancer with a combination of an antibody-drug conjugate and an androgen receptor antagonist, whereby the antibody recognizes an extracellular epitope of CDCP1, whereby the drug conjugate is a chemotherapeutic drug, a toxic compound or a radioactive compound, and whereby the anti-androgen preferably is enzalutamide.

A downmodulator of CDCP1 for use according to the invention may be combined with anti-androgen therapy. The combination with anti-androgen therapy results in a continuous upregulation of CDCP1 in prostate cancer cells, which cells become senescence by co-administration of the downmodulator of CDCP1. A person skilled in the art will understand that the anti-androgen therapy preferably precedes, at least in part, the administration of the downmodulator of CDCP1. The means that the combination results in a consecutive, potentially overlapping, administration of the anti-androgen and the downmodulator of CDCP1, whereby the start of administration of the anti-androgen is at least one day before the start of administration of the downmodulator of CDCP1.

Said anti-androgen therapy preferably is or comprises an androgen receptor antagonist, selected from enzalutamide at 50-300 mg orally, once daily, preferably about 160 mg orally, once daily; bicalutamide at 25-300 mg orally, once daily, preferably about 150 mg orally, once daily, and nilutamide 100-450 mg orally, once daily, preferably about 150 mg orally, once daily.

A downmodulator of CDCP1 for use according to the invention preferably is combined with a senolytic compound. Said combination with a senolytic compound selectively induces death of castrate-resistant prostate cancer cells that have become senescent.

A downmodulator of CDCP1 for use according to the invention preferably is combined with a genotoxic agent such as an alkylating agent such as nitrogen mustard, e.g. cyclophosphamide, mechlorethamine or mustine, uramustine and/or uracil mustard, melphalan, chlorambucil, ifosfamide; nitrosourea, including carmustine, lomustine, streptozocin; an alkyl sulfonate such as busulfan, an ethylenime such as thiotepa and analogues thereof, a hydrazine/triazine such as dacarbazine, altretamine, mitozolomide, temozolomide, altretamine, procarbazine, dacarbazine and temozolomide; an intercalating agent such as a platinum-based compound like cisplatin, carboplatin, nedaplatin, oxaliplatin and satraplatin; anthracyclines such as doxorubicin, daunorubicin, epirubicin and idarubicin; mitomycin-C, dactinomycin, bleomycin, adriamycin, and mithramycin.

A downmodulator of CDCP1 for use according to the invention preferably is combined with anti-androgen therapy, preferably an androgen receptor antagonist, and a senolytic compound.

Said senolytic compound preferably is selected from rapamycin (0.1-20 mg daily, preferably about 2 mg daily), acarbose (5-150 mg daily, preferably about 50 mg daily), nordihydroguaiaretic acid (NDGA; (500-5000 mg daily, preferably about 2000 mg daily), ABT263 (10-500 mg daily, preferably about 150 mg daily), FOXO4-DRI (0.2-50 mg daily or every second day, preferably about 20 mg daily or every second day, a CXCR4 inhibitor and/or antagonist such as TG-0054 (2-[4-[6-amino-2-[[4-[[3-(cyclohexylamino)propylamino]methyl]cyclohexyl]methylamino]pyrimidin-4-yl]piperazin-1-yl]ethylphosphonic acid), AMD070 (˜{N}′-(1˜{H}-benzimidazol-2-ylmethyl)-˜{N}′-[(8˜{S})-5,6,7,8-tetrahydroquinolin-8-yl]butane-1,4-diamine), AMD3100 or plerixafor (1-[[4-(1,4,8,11-tetrazacyclotetradec-1-ylmethyl)phenyl]methyl]-1,4,8,11-tetrazacyclotetradecane; 0.5-50 mg daily), cyclic peptide CXCR4 antagonists such as LY2510924, and dasatinib (10-500 mg daily, preferably about 140 mg daily).

The administration of the senolytic compound preferably is started one day, preferably two days, preferably 3 days, preferably more than 3 days such as 1-2 weeks after the start of the administration of a downmodulator of CDCP1.

The invention further provides a method of treating a patient with prostate cancer with a combination of a downmodulator of CDCP1 and a senolytic compound, the method comprising determining if a level of testosterone in a bodily fluid of the patient; identifying a patient of which the level of testosterone is below 50 ng/dL;

determining whether the prostate cancer is progressing in the identified patient; and treating a patient in which the testosterone level is below 50 ng/dL and in which prostate cancer is progressing as a patient with a combination of a downmodulator of CDCP1 and a senolytic compound.

The invention further provides a method of treating a patient with castrate-resistant prostate cancer, comprising identifying a patient who suffers from castrate-resistant prostate cancer; and treating said identified patient with a combination of a downmodulator of CDCP1 and a senolytic compound.

In a preferred method of treating according to the invention, the downmodulator is administered prior to the administration of the senolytic compound.

### 4.6 Compositions

The invention further provides a pharmaceutical composition, comprising a downmodulator of CDCP1 and a senolytic compound and/or a genotoxic agent.

Said pharmaceutical composition for preferably is a sterile isotonic solution. Said buffer preferably is citrate-based buffer, preferably lithium-, sodium-, potassium-, or calcium-citrate monohydrate, citrate trihydrate, citrate tetrahydrate, citrate pentahydrate, or citrate heptahydrate; lithium, sodium, potassium, or calcium lactate; lithium, sodium, potassium, or calcium phosphate; lithium, sodium, potassium, or calcium maleate; lithium, sodium, potassium, or calcium tartarate; lithium, sodium, potassium, or calcium succinate; or lithium, sodium, potassium, or calcium acetate, or a combination of two or more of the above. The pH of said buffer may be adjusted, preferably to a pH of 7.27-7.37 by hydrochloric acid, sodium hydroxide, citric acid, phosphoric acid, lactic acid, tartaric acid, succinic acid, or a combination of two or more of the above. The volume of may range from 0.5 ml to 5 ml. Said excipient preferably is selected from, but not limited to, urea, L-histidine, L-threonine, L-asparagine, L-serine, L-glutamine, polysorbate, polyethylene glycol, propylene glycol, polypropylene glycol, or a combination of two or more of the above.

A pharmaceutical composition according to the invention preferably comprises a kit of parts, comprising a pharmaceutical preparation comprising the downmodulator of CDCP1 and a pharmaceutical preparation comprising the senolytic compound and/or a genotoxic agent. Said kit of parts preferably further comprising instructions for administration of a downmodulator of CDCP1 prior to the administration of a the senolytic compound, preferably one day, preferably two days, preferably 3 days, preferably more than 3 days such as 1-2 weeks prior to the administration of a the senolytic compound.

A further preferred pharmaceutical composition comprises liposomes as described herein above, said liposomes comprising the downmodulator of CDCP1. Said liposomes preferably further comprise a genotoxic agent, preferably an anthracycline such as doxorubicin.

A pharmaceutical composition according to the invention preferably is for use in a method of treating a patient suffering from castrate-resistant prostate cancer.

### 4.7 Method of Selecting a Patient Who is Eligible for Treatment with a Combination of a Downmodulator of CDCP1 and a Senolytic Compound

The invention further provides a method of selecting a patient with prostate cancer eligible for treatment with a combination of a molecule that downmodulates expression of CUB domain-containing protein 1 (CDCP1) on the surface of prostate cancer cells and a senolytic compound, comprising determining if a level of testosterone in a bodily fluid of the patient; identifying a patient of which the level of testosterone is below 50 ng/dL; determining whether the prostate cancer is progressing in the identified patient; and selecting a patient in which the testosterone level is below 50 ng/dL and in which prostate cancer is progressing as a patient who is eligible for treatment with a combination of a downmodulator of CDCP1 and a senolytic compound.

Said progression of prostate cancer preferably is determined by a continuous rise in serum prostate-specific antigen (PSA) levels, and/or the appearance of new metastases in said patient.

### 5 EXAMPLES

### Example 1

### Materials and Methods

Mice

All mice were maintained under specific-pathogen-free conditions in the animal facilities of the Institute for Research in Biomedicine, Bellinzona and experiments were performed according to state guidelines and approved by the local ethics committee. The PtenloxP conditional knockout alleles have been described (Chen, et al., 2005. Nature 436: 725-730). CDCP1 conditional overexpression were generated as described in the results. To obtain all the gentypes, female CDCP1 and/or PtenloxP/loxP mice were crossed with male Probasin-Cre4 (Pb-Cre4) transgenic mice (Trotman et al., 2003. PLoS Biol 1, E59) for the prostate-specific overexpression of CDCP1 and deletion of Pten. For genotyping, tail DNA was subjected to polymerase chain reaction analysis with the following primers. For PtenloxP/loxP, primer 1 (5′-AAAAGTTCCCCTGATGATGATTTGT-3′) and primer 2 (5′-TGTTTTTGACCAATTAAAGTAGGCTGTG-3′) were used. To detect the deleted allele in prostate, primer 3 (5′-TTCTCTTGAGCACTGTTTCACAGGC-3′) and primer 1 were used. For Probasin-Cre4 (Pb-Cre4), primer 1 (5′-TGATGGACATGTTCAGGGATC-3′) and primer 2 (5′-GCCACCAGTCTGCATGA-3′) were used in genotyping or detecting the allele in prostate. For CDCP1 mice, primer 1 (5′-CAAGGGAGAAGAGAGTGCGG-3′) and primer 2 (5′-CCCAACAATGGGGATGTAAG-3′) were used.

For the xenograft experiments, 1×10⁶ PC3 cells infected Tripz-shCDCP1 or Tripz-shRNA control were injected subcutaneously (s.c.) of SCID-NOD. The mice started to be fed with Doxycycline (0.2 g/L) water supplemented with 5% sucrose upon tumour onset. Tumour formation from each individual injection was monitored every three days until experimental termination Animals were autopsied, and all tissues were examined regardless of their pathological status. Normal and tumour tissue samples were fixed in 10% neutral-buffered formalin (Catalog #HT501128, Sigma) overnight. Tissues were processed by ethanol dehydration and embedded in paraffin according to standard protocols. Sections (5 μm) were prepared for antibody detection and haematoxylin and eosin staining

Cell Culture and Reagents

Human prostate carcinoma cell lines, including PC3, were purchased from ATCC and were cultured according to the manufacturer's instructions. Cells were transduced with PLKO or TRIPZ doxycycline inducible lentiviral construct against human CDCP1 gene and empty Vector obtained by Thermo Scientific, Waltham, Mass., USA (CloneIDs: V3THS_329377 and V2THS_191307). LNCaP-abl and LAPC4 cells were a gift from Dr. Jean-Philippe Theurillat (Institute of Oncology Research (IOR), Bellinzona). PC3-AR were generated by infecting them with retroviruses expressing full-length human AR (provided by Dr. Jean-Philippe Theurillat. PC3-AAR were generated using the expression of human AR with the deletion of amino acids 538-614, deletion of AR DNA binding domain (Addgene, Catalog #89107). LNCaP-ADI cells were generated from parental LNCaP by growing them in RPMI 1640 containing 10% charcoal-stripped FBS. Androgen stimulation experiments were performed using 1 nM of the 5α-Dihydrotestosterone (DHT) (Sigma, Catalog #521-18-6). Full androgen deprivation (FAD) experiment was performed culturing the cells in RPMI with Charcoal-stripped FBS and Enzalutamide. Enzalutamide (APExBIO Catalog #A3003) was dissolved in DMSO at a concentration of 10 uM.

Primary MEFs were derived from littermate embryos and obtained by crossing CDCP1; PtenWT/loxP with PtenWT/loxP animals. Embryos were harvested at 13.5 days postcoitum, and individual MEFs were produced and cultured as previously described (Chen et al., 2005. Nature 436: 725-730). Primary MEFs were infected with retroviruses expressing pMSCV-CRE-PURO-IRES-GFP for 48 h and selected with Puromycin at a concentration of 2 μg/mL. Briefly, all genotypes MEFs were obtained by crossing male wild type and Ptenlox-lox with female CDCP1lox-stop-lox mice. A pregnant mouse at 13- or 14-day post-coitum was sacrificed by cervical dislocation. Embryos were harvested and the individual MEFs were cultured in DMEM containing 10% fetal bovine serum and 1% PenStrep. Primary Ptenlox/lox MEFs were infected with retroviruses expressing either pMSCV-CRE-PURO-IRES-GFP or pMSCV-PURO-IRES-GFP for 48 h and selected with Puromycin at a concentration of 3 ug ml and as previously described. All mice were maintained under specific pathogen-free conditions in the animal facilities of the IRB institute, and the experiments were performed according to the state guidelines and approved by the local ethical committee.

The following antibodies were used for western blotting: Tag-Myc (Catalog #551101; BD Pharmingen, 1:1000); tubulin (DSHB, E7, 1:1000); pCDCP1(Catalog #13111; Cell Signaling Technology, 1:1000); PTEN (Catalog #95525; Cell Signaling Technology, 1:1000); HSP90 (Catalog #48775; Cell Signaling Technology, 1:1000); c-Myc (Catalog #A713(G-4), Santa Cruz Biotechnology, 1:500); p21 (Catalog #F1013(C-19), Santa Cruz Biotechnology, 1:500); 6-actin (Catalog #A5316; Sigma, 1:5000); Cyclin D1 (Catalog #29785, Cell Signaling Technology, 1:1000); COUP-TFII (Catalog #PP-H7147-00; Perseus Proteomics, 1:1000). SMAD4 (Catalog #E0615, Santa Cruz Biotechnology, 1:500). p-SRC-Tyr416 (Catalog #69435, Cell Signaling Technology, 1:1000); SRC (Catalog #2123S, Cell Signaling Technology, 1:1000); AKT (Catalog #92725, Cell Signaling Technology, 1:1000); p-AKT-5473 (Catalog #9171S, Cell Signaling Technology, 1:1000); p53 (Catalog #MEDRPRO25-1, Accuratechemical, 1:1000); CDCP1 (Catalog #4115, Cell Signaling Technology, 1:1000); Erk1/2 (Catalog #46955, Cell Signaling Technology, 1:1000). p-Erk1/2-Thr202/Tyr204 (Catalog #9106S, Cell Signaling Technology, 1:1000); S6 (Catalog #2317S, Cell Signaling Technology, 1:1000); pS6-Ser235/236 (Catalog #4857, Cell Signaling Technology, 1:1000); p27 (Catalog #K0413, Santa Cruz Biotechnology, 1:500); AR (N-20) (Catalog #SC-816 Santa Cruz Biotechnology, 1:500).

For IHC the following antibodies were used: Ki-67 (clone SP6, Catalog #RT-9106-R7; Rabbit Polyclonal; Unmask Watherbath 98° C. pH6 20′; Lab Vision, Dilution Ready To Use); CDCP1 (Catalog #4115, Rabbit Polyclonal; Unmask Watherbath 98° C. pH6 20′; Cell Signaling Technology, 1:50); p-HPly-Ser83 (Catalog #2600, Unmask Watherbath 98° C. pH6 20′; Cell Signaling Technology, 1:50), Cyclin D1 (Catalog #29785, Cell Signaling Technology); AR (N-20) (Catalog #SC-816, Rabbit Poly; Unmask Watherbath 98° C. pH6 20′; Santa Cruz Biotechnology, 1:300); Wide Spectrum Cytokeratin (Pankeratin) (Catalog #Z0622; Rabbit Polyclonal; Unmask Watherbath 98° C.pH9 20′; DAKO, 1:2000).

For IF the following antibodies were used: E Cadherin (Clone 26) (Catalog #610181; Mouse Monoclonal; Unmask Watherbath 98° C.pH9 20′; BD, 1:700); CK5 (Catalog #ab52635; Rabbit Polyclonal; Unmask Watherbath 98° C.pH9 20′; Abeam, 1:500); CK8 (Catalog #ab59400; Rabbit Polyclonal; Unmask Watherbath 98° C.pH9 20′; Abeam, 1:150), CDCP1 (Catalog #4115, Cell Signaling Technology, 1:100) and DE-cadherin (Developmental Studies Hybridoma Bank [DSHB], DCAD2, 1:100). c-Myc siRNA and negative control siRNA were purchased from Sigma (Catalog #8024873724-000050; #8024873724-000060). We transfected the cells with Lipofectamin RNAiMAX (Catalog #13778-030; Invitrogen) according to manufacture's protocol.

Generation of GAL4-UAS-CDCP1-wt and GAL4-UAS-CDCP1-delta Drosophila melanogaster lines and Immunofluorescence (IF) UAS-egfr.B (5368), src64BP1 (7379), Src42AK10108 (10969), GMR-gal4 (1104) and ptc-gal4 (2017) lines were obtained from Bloomington Drosophila Stock Centre. Cultures were carried out on a cornmeal/agar diet, (6.65% cornmeal, 7.15% dextrose, 5% yeast, 0.66% agar supplemented with 2.2% nipagin and 3.4 ml 1-1 propionic acid). Cultures were performed at 25° C. and 29° C. To overexpress human CDCP1-wt and CDCP1-delta, UAS transgenic lines were generated from human CDCP1-wt and CDCP1-delta cDNA with the following primer pair: (5′-GATATCCACCATGGCCGGCCTGAACTGCGGG-3′) and (5′-ACTAGTTCAATGGTGATGGTGATGATG-3′). PCR was performed with Q5 high-fidelity polymerase from New England Biolabs (M0491S). PCR products were cloned using Zero Blunt TOPO PCR Cloning Kit (Life Technologies, K2800-20) before cloning into pUAST-attB vector (Bischof et al., 2007. PNAS USA 104: 3312-3317). The constructs were sequence-verified and the transgenic lines established through PhiC31 integrase-mediated transgenesis (BestGene, attP site: VK27). Salivary glands were dissected in PBS, fixed in 4% paraformaldehyde (PFA) in PBS, washed in PBT (PBS containing 0.1% Triton X-100) and incubated with primary antibodies in PAXDG (PBS containing 1% BSA, 0.3% Triton X-100, 0.3% deoxycholate, and 5% goat serum) overnight at 4° C. Tissue was washed with PBT and incubated with secondary antibodies in PAXDG for 5 h at 4° C. and mounted in Vectashield mounting media (Vector Laboratories). Alexa-568-conjugated anti-rabbit and Alexa-488-conjugated anti-rat antibodies were used as secondary antibodies (Molecular Probes). Images of adult eye and bristle were taken with a Leica M165 FC microscope equipped with SXY-I30 3M Pixel Colour Camera. Fluorescent images of salivary glands were taken with Leica M165 FC fluorescent microscope equipped with Leica DFC 3000G digital camera.

CDCP1 Protein Expression in Human Prostate Cancer

The first TMA was composed of benign prostate tissue and PCa at different stages (n=237), as previously reported (Zellweger et al., 2013. Endocr Relat Cancer 20: 403-413). Spots with metastases were not included in the analysis to avoid false negative results due to poor fixation of tissue (mostly material from autopsies). The second TMA (n=192) consisted of locally advanced, inoperable, mostly metastatic PCa including CRPC and hormone naïve (untreated) PCa, as previously reported (Zellweger et al., 2013. Endocr Relat Cancer 20: 403-413). For distant metastasis CDCP1 staining was performed on 6 regular histological sections of distant and lymph node PCa metastases. H-Score: intensity of staining on a scale of 0 (no staining), 1+(weak staining), 2+(moderate staining), and 3+(strong staining) multiplied by the percentage of positive tumour cells. Cytoplasmic staining only was considered negative. Contingency table analysis and chi-square tests were used to study the relationship between the marker expression and histological subgroups. Differences between values were considered statistically significant with a p value of <0.05. The analyses were performed with JMP 12 software (SAS Institute, Cary, N.C., USA). Use of the clinical samples for TMA construction was approved by the Ethical Committee of the University of Basel.

Paired Diagnostic (HSPC) and CRPC Biopsies

Patients were identified from a population of men with metastatic castration resistant prostate cancer (CRPC) treated at the Royal Marsden NHS Foundation Trust. All patients had given written informed consent and were enrolled in institutional protocols approved by the Royal Marsden NHS Foundation Trust Hospital (London, UK) ethics review committee (reference no. 04/Q0801/60). Twenty-five patients with a diagnosis of prostate adenocarcinoma with sufficient formalin-fixed, paraffin-embedded (FFPE), matched diagnostic (archival) hormone sensitive prostate cancer (HSPC) and CRPC tissue for CDCP1 immunohistochemistry were selected. HSPC tissue demonstrated adenocarcinoma and was obtained from either prostate needle biopsy (21 cases), transurethral resection of prostate (TURP; 3 cases) or bone biopsy (1 case). CRPC tissue was obtained from the same patients through biopsies of bone (19 cases), lymph node (5 cases) or liver (1 case). All tissue blocks were freshly sectioned and only considered for IHC analyses if adequate material was present (>50 tumour cells; reviewed by Daniel Nava Rodrigues).

Correlation Analysis

Correlation between CDCP1 and PTEN in PCa data sets (Taylor et al., Cancer Cell 18: 11-22; Chandran et al., 2007. BMC Cancer 7: 64; Varambally et al., 2005. Cancer Cell 8: 393-406; Grasso et al., 2012. Nature 487: 239-243; Lefort et al., 2016. Oncotarget 7: 48011-48026) was carried out using Spearman's correlation which estimates a correlation coefficient value ‘R’ and a significance P value.

Immunoblotting

Tissue and cell lysates were prepared with RIPA buffer (Catalog #9806, Cell Signaling Technology) with PMSF (Phenylmethanesulfonyl fluoride; Catalog #329-98-6, Sigma). Protein concentrations of the lysates were measured by Pierce BCA Protein Assay Kit (Catalog #23225, Thermo Scientific). The lysates were then resolved by SDS-PAGE and immunoblotted with the indicated antibodies. For analysis of fly tissue, wandering third-instar larvae were rinsed in PBS, salivary glands were dissected out, washed in PBS and homogenised in SDS sample buffer.

Real-Time PCR

RNA was extracted using TRIzol® Plus RNA Purification Kit (Catalog #12183555, Life technologies). 1 μg of total RNA was used for cDNA synthesis using SuperScript® III Platinum® One-Step qRT-PCR Kit (Catalog #11732-020, Life technologies). Quantitative Real Time PCR (q-RT PCR) was performed as previously described (Chen et al., 2005. Nature 436: 725-730). Primers employed are listed in Tables 1 and 2. All qRT-PCR data presented was normalized using GAPDH, HRPT or 18S rRNA.

TABLE 1
Primers for RT-PCR (Mouse)

| | |
|---|---|
| p16Ink4a forward | 5′-CGCAGGTTCTTGGTCACTGT-3′ |
| p16Ink4a reverse | 5′-TGTTCACGAAAGCCAGAGCG-3′ |
| p27 forward | 5′-GCAAAACAAAAGGGCCAACA-3′ |
| p27 reverse | 5′-GGGCGTCTGCTCCACAGT-3′ |
| Gapdh forward | 5′-AGGTCGGTGTGAACGGATTTG-3′ |
| Gapdh reverse | 5′-TGTAGACCATGTAGTTGAGGT-3′ |
| Pten forward | 5′-TGGATTCGACTTAGACTTGACCT-3′ |
| Pten reverse | 5′-GCGGTGTCATAATGTCTCT-3′ |
| Rn18S forward | 5′-ACCGCAGCTAGGAATAATG-3′ |
| Rn18S reverse | 5′-GCCTCAGTTCCGAAAACCA-3′ |
| COUP-TF II forward | 5′-TCAACTGCCACTCGTACCTG-3′ |
| COUP-TF II reverse | 5′-CATGATGTTGTTAGGCTG-3′ |
| Cyclin D1 forward | 5′-GCGTACCCTGACACCAATC-3′ |
| Cyclin D1 reverse | 5′-CTCCTCTTCGCACTTCTGCTC-3′ |
| c-Myc forward | 5′-CTGGACCAGGGAGTGGAGT-3′ |
| c-Myc reverse | 5′-ACGTAGTAGTCGGTTCTCA-3′ |
| Fkbp5 (Biorad) | (Catalog #10041595 |
| | PrimePCR™ PreAmp for |
| | SYBR® Green Assay) |
| PSCA (Biorad) | (Catalog #10041595 |
| | PrimePCR™ PreAmp for |
| | SYBR® Green Assay) |

ChIP Assay

Cells were cultured up to a confluence of 90-95% and were crosslinked with 1% formalin for 10 min followed by addition of 2.5 M glycine for 5 min at room temperature. The culture medium was aspirated and the cells were washed twice with ice-cold phosphate-buffered saline. Nuclear extracts were sonicated using a Misonix 3,000 model sonicator to sheer crosslinked DNA to an average fragment size of ˜500 base pairs. Sonicated chromatin was incubated for 16 h at 4° C. with γ-bind Plus Sepharose beads (Catalog #17-0886-01, GE Healthcare) conjugated with either anti-c-Myc ((9E10)×L0815) anti-SMAD4 ((B-8) Catalog #E0615; Santa Cruz) or Mouse-IgG antibody (Catalog #92590 Millipore) by incubating overnight at 4° C. on a rotor. After incubation, beads were washed thoroughly and then centrifuged. The chromatin was eluted from the beads, and crosslinks were removed by incubation at 56° C. for 12 h. DNA was then purified using the QIAquick PCR Purification Kit (Catalog #28104, Qiagen). The binding of the transcription factor, c-Myc, on Cyclin D1 promotor was determined using SABiosciences' proprietary database DECipherment Of DNA Elements (DECODE).

TABLE 2
Primers for RT-PCR (Human)

| | |
|---|---|
| p21 forward | 5′-TGTCCGTCAGAACCCATGC-3′ |
| p21 reverse | 5′-AAAGTCGAAGTTCCATCGCTC-3′ |
| p27 forward | 5′-TAATTGGGGCTCCGGCTAACT-3′ |
| p27 reverse | 5′-TGCAGGTCGCTTCCTTATTCC-3′ |
| GAPDH forward | 5′-AATCCCATCACCATCTTCCA-3′ |
| GAPDH reverse | 5′-TGGACTCCACGACGTACTCA-3′ |
| c-MYC forward | 5′-CGGAACTCTTGTGCGTAAGG-3′ |
| c-MYC reverse | 5′-CTCAGCCAAGGTTGTGAGGT-3′ |
| CDCP1 forward | 5′-TGGTTCCACCCCAGAAATGT-3′ |
| CDCP1 reverse | 5′-GATGATGCACAGACGTTTTAT-3′ |
| SLC45A3 forward | 5′-TCGTGGGCGAGGGGCTGT-3′ |
| SLC45A3 reverse | 5′-CATCCGAACGCCTTCATC-3′ |
| TMPRSS2 forward | 5′-TACTCTGGAAGTTCATGG-3′ |
| TMPRSS2 reverse | 5′-GTCATCCACTATTCCTTG-3′ |
| KLK2 forward | 5′-CTGCCCATTGCCTAAAGA-3′ |
| KLK2 reverse | 5′-GTAGAGCGGGTGTGG-3′ |
| KLK3PSA forward | 5′-GGAAATGACCAGGCC-3′ |
| KLK3PSA reverse | 5′-CCAGCTTCTGCTCAGTGC-3′ |

The primer mix used for ChIP assay was EpiTect ChIP qPCR Primer Assay For Mouse Ccnd1, NM_007631.2 (−)04 Kb (Catalog #GPM1053924(−)04A). The primer sequences designed for the ChIP assay to 1) detect Smad4 binding site (SBE) on Cyclin D1 promotor were: SBEChIPf 5′-CCGCTTAGTCCCCATTCTAAAG-3′ and SBEChIPr: 5′-GGCATCTCCATTCTTAATCCAG-3′; 2) detect c-Myc binding on Coup-tfII promotor is: COUP-TFIIChIPf 5′-GTGCGGGGACAAGTCGAGCGG-3′ and COUP-TFIIChIPr 5′-GCGGTGGTGCTGGTCGATGGG-3′. ChIP qPCR was performed using KAPA SYBR FAST ABI qPCR Master Mix solution (Catalog #07959389001, KAPA Biosystem, Roche) on Step One Real-Time PCR systems (Applied Biosystems).

Proliferation and Senescence Assays

Proliferation assay in MEFs was performed by plating 10⁴ cells per well of 24-well plate in triplicate while that in human PCa cell lines was performed by plating 1-2×10⁴ cells per well of 24-well plate in triplicate. Cell proliferation was monitored on days 0, 3, 6 and 9 whereby cells were fixed for 15 min in a solution of 10% buffered formalin washed with phosphate-buffered saline (pH7.2) and subsequently stained with 0.01% Crystal Violet solution. Excessive staining was removed by washing with distilled water and drying the plates overnight. Crystal violet-stained cells were dissolved in 10% acetic acid solution for 30 min on a shaker and the extracted dye was read with a spectrophotometer at 590 nm. Cellular senescence in vitro was performed using the Senescence ß-Galactosidase Staining Kit (Catalog #9860; Cell Signaling) as per the manufacturer's instructions and the quantification were done counting the total number of cells with Hoechst 3342, Trilhydrochloride, Trihydrate (Catalog #953557; Invitrogen).

Stealth Liposomes

Stealth liposomes were prepared using HSPC:CHOL:mPEG5 kDa-DSPE at a 18:9:1 molar ratio. The lipid film, obtained evaporating a chloroform solution of the components, was hydrated with a solution of 250 mM ammonium sulfate (pH 5.5) and then extruded at 60° C. until reaching the vesicle size of ˜100 nm. The external buffer was exchanged to PBS pH 7.4 by a PD-10 desalting column. Doxorubicin (DXR) was encapsulated by remote loading (DXR:HSPC 0.2:1 w/w) at 60° C. Free DXR was removed using a PD-10 desalting column and the drug loading was determined spectrophotometrically (=477 nm) in methanol. The CUB4 Fab′-coupled PEG-phospholipid derivative was prepared by reacting the Fab′ of CUB4, obtained as described below, with maleimide-PEG-DSPE and then CUB4 Fab′-PEG-DSPE was introduced on the liposome surface by post-insertion to provide stealth immunoliposomes (SIL). Briefly, CUB4 was enzymatically digested with pepsin (1:50 w/w E/S, 3 h at 37° C.) in 0.1 M sodium acetate at pH 3.8, followed by FPLC analysis on a Superose 12 10/300 GL column using PBS pH 7.4 (fow-rate 0.5 ml/min). The F(ab′)2 fragment was collected and treated 30 minutes at rt with 10 mM cysteamine to yield the Fab′ fragment, following purified by FPLC using 50 mM phosphate buffer, 150 mM NaCl and 10 mM EDTA, pH 5. By exploiting its free sulfhydryl groups, Fab′ was immediately coupled (overnight at rt, pH 7.0-7.5) to the maleimide groups of mixed micelles composed of Maleimide-PEG5 kDa-DSPE:mPEG5 kDa-DSPE 4:1 mol/mol at a final molar ratio of 10:1 Maleimide:Fab′. Finally, these micelles were incubated 1 h at 60° C. with SL at a molar ratio of 0.05:1 PEG:HSPC to achieve SIL, followed by purification on a Sepharose CL-4B column using PBS pH 7.4 and Fab′ quantification by BCA assay.

Statistics

All data points are presented for quantitative data, with an overlay of the mean with SD and SEM (specified in the figures legends). All statistical analysis were performed using Graph Pad Prism 8 or Microsoft Excel 2016 or R-studio. The statistic test used is the T-test 1 or 2 tailed (as specified in the figures legends). Other used statistical analysis were indicated in the figure legends.

### Results

CRPC and Metastatic Prostate Tumors Exhibit Elevated Expression of CDCP1 and Overexpression of CDCP1 Correlate with PTEN Loss

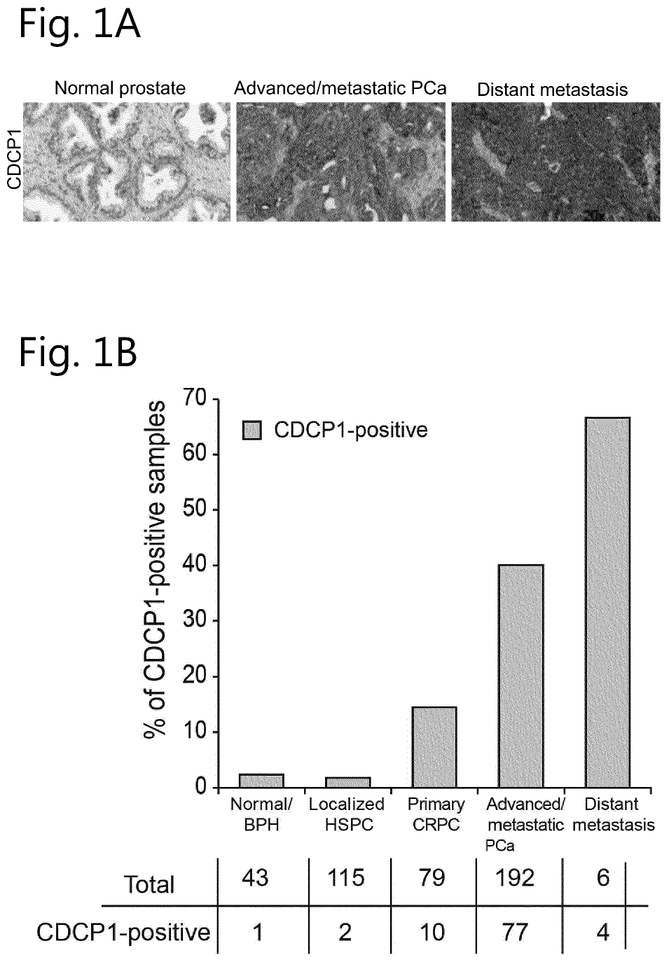

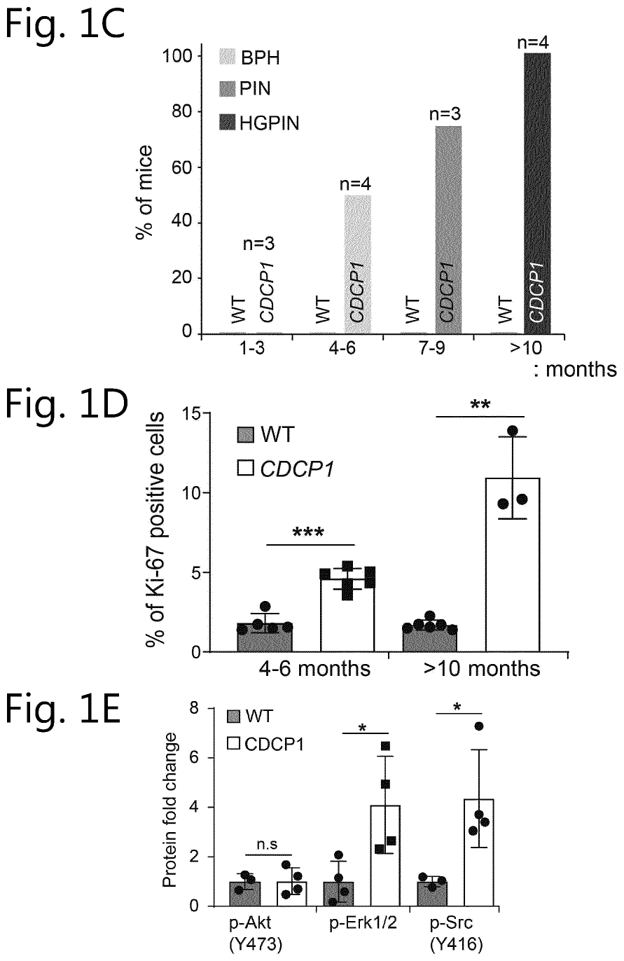

CUB domain-containing protein 1 (CDCP1) is a transmembrane protein that acts as a substrate for Src family tyrosine kinases (SFKs) which can be inhibited by either monoclonal antibodies or small molecule inhibitors (Nakashima et al., 2017. Cancer Sci 108: 1049-1057; Kollmorgen et al., 2013. Mol Oncol 7: 1142-1151; Siva et al., 2008. Cancer Res 68: 3759-3766). To assess whether CDCP1 is overexpressed in human prostate cancer (PCa), we evaluated CDCP1 protein expression in different tissue microarrays (TMAs), including 435 PCa cases of benign, locally advanced and metastatic PCa. Immunohistochemical (IHC) analyses showed that CDCP1 was mainly overexpressed in advanced and metastatic tumor samples from CRPC patients, when compared to benign prostate tumours from hormone sensitive patients (FIG. 1A, B). While these data pointed at CDCP1 as a relevant oncogene, recent findings in different tumor models show that CDCP1 acts as a tumor suppressor in cancer. To validate the oncogenic potential of CDCP1 we generated a transgenic mouse model overexpressing human CDCP1 in the mouse prostate and a Drosophila melanogaster model overexpressing both a normal and a mutant form of human CDCP1.

At first, we constructed a pGACCS vector with a transcriptional STOP sequence flanked by loxP sites upstream of CDCP1-cDNA. The resulting PGACCS-loxP-STOP-loxP-CDCP1-vector along with PGK-FlpO plasmid were co-electroporated into the ColA locus modified embryonic stem cells KH2 (Beard et al., 2006. Genesis 44: 23-28; data not shown). PCR and Southern-blot analysis confirmed gene integration and recombination events (data not shown), whereas western blot analysis confirmed the expression of CDCP1 in mouse embryonic fibroblasts (MEFs) derived from these mice4 (data not shown). Thereafter, we crossed CDCP1 animals with PB-Cre4 mice for prostate-specific expression of CDCP1. Of note, the expression of CDCP1 in a panel of human prostate tumor cell lines, patient-derived prostate cancer xenografts (PDXs) and tumors collected from CDCP1+ mice did not show significant differences in the CDCP1 levels (data not shown) thereby demonstrating that overexpression of CDCP1 in the mouse model is similar to the CDCP1 levels in human tumors. IHC analyses were performed on 10-weeks old prostate tissues of CDCP1; Pb-Cre mice (CDCP1pcLSL/+, hereafter referred to as CDCP1) to confirm the prostate-specific expression of CDCP1 (data not shown). To note, CDCP1 mRNA and protein levels were highly expressed in the anterior prostate (AP) and the ventral prostate (VP) when compared to dorsolateral prostate (DLP) (data not shown). Next, we examined tumour incidence in the CDCP1 mouse model over a period of 24 months. In cohorts of mice, we observed prostate hyperplasia between 4-6 months of age with 50% penetrance and prostatic intraepithelial neoplastic (PIN) lesions, characterized by prostatic glands containing multilayers of epithelial cells with features of nuclear atypia (75% penetrance) between 7-9 months of age. The CDCP1 mice developed high grade PIN (HGPIN) after 14 months of age with 100% penetrance (FIG. 1C) in the anterior, dorso-lateral and ventral prostates (data not shown). A significant increase in proliferation of the prostatic epithelium of CDCP1 mice was observed by Ki-67 positive staining, as compared to their wild-type age-matched littermates (FIG. 1D). In parallel, western blot analysis revealed a significant increase of Src and Erk1/2 phosphorylation in the prostatic epithelium and in MEFs overexpressing CDCP1 (FIG. 1E).

We next overexpressed wildtype human CDCP1 (CDCP1-WT) and CDCP1-delta, a mutant form of human CDCP1 lacking Src-phosphorylation sites (CDCP1-delta), in Drosophila melanogaster (Alajati et al., 2015. Cell Rep 11: 564-576; Liu et al., 2011. PNAS 108: 1379-1384). The Drosophila larval imaginal discs are a monolayer epithelium that is considered morphologically comparable to mammalian epithelia and therefore constitutes an ideal system in which to model cancer progression in vivo (Brumby and Richardson, 2005. Nat Rev Cancer 5: 626-639). Increased EGFR/Ras signaling has been previously shown to promote the formation of bristles located on the dorsal part of the fly thorax (notum) (also referred to as macrochaetae formation) a tumor-like phenotype (Culi et al., 2001. Development 128: 299-308; Khare et al., 2017. PLoS One 12: e0173565). We found that overexpression of CDCP1-WT, but not CDCP1-delta, promoted extra-macrochaetae formation of the fly notum (data not shown). Interestingly, loss of one copy of both src42A and src64B (50% reduction) suppressed extra-macrochaetae formation driven by the overexpression of CDCP1-WT, demonstrating that this phenotype is Src dependent (data not shown). Moreover, over-expression of CDCP1 triggered aberrant proliferation in the fly eye (data not shown). Collectively, these results using a cross-species genetic approach, demonstrate that CDCP1 overexpression in vivo initiates tumorigenesis by activating the Src-MAPK signaling pathway.

CDCP1 Cooperates with Pten-Loss to Drive Prostate Cancer Progression and Metastatic Castration Resistant Prostate Cancer

To further assess the role of CDCP1 as driver of CRPC we intercrossed the CDCP1 mice with the Pten-null prostate conditional mice (Pten^{pc-/-}) that develop castration sensitive prostate tumors and we obtained the CDCP1; Pten^{pc-/-} double mutant mice. While monoallelic loss or mutations in PTEN is associated with benign prostate tumors (Trotman et al., 2003. PLoS Biol 1: E59; Alimonti et al., 2010. Nat Genet 42: 454-458), complete loss of PTEN is frequently observed in human metastatic prostate cancer (Taylor et al., 2010. Cancer Cell 18: 11-22). However, complete loss of Pten in the mouse is not sufficient to promote metastatic prostate cancer and additional genetic hits are needed to promote the onset of metastases (Chen et al., 2005. Nature 436: 725-730).

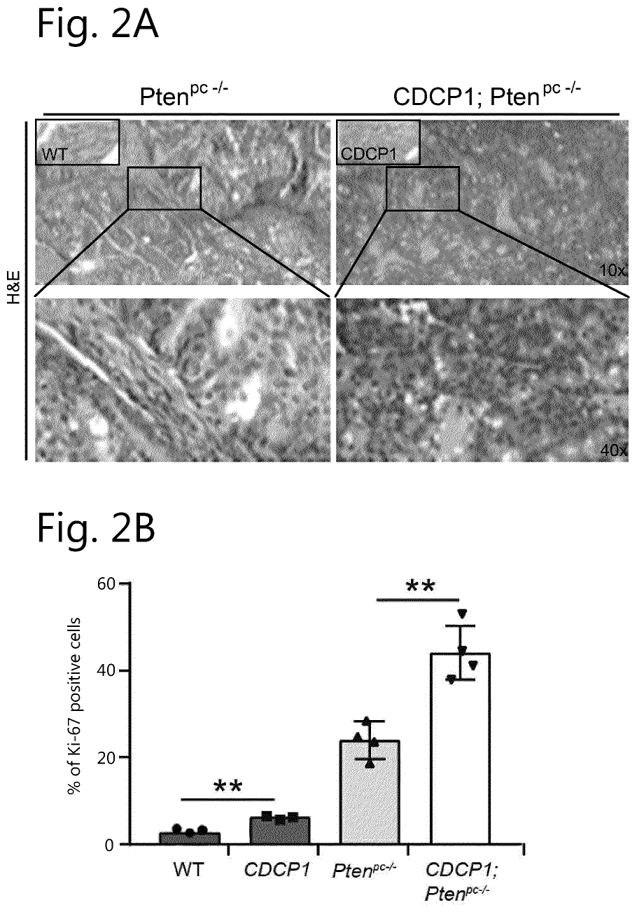

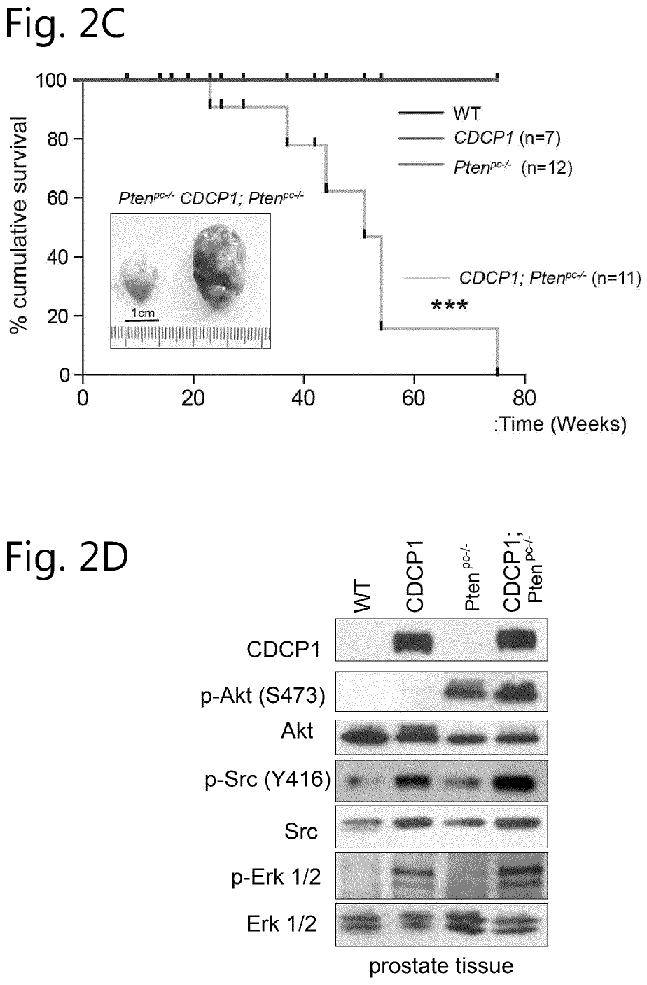

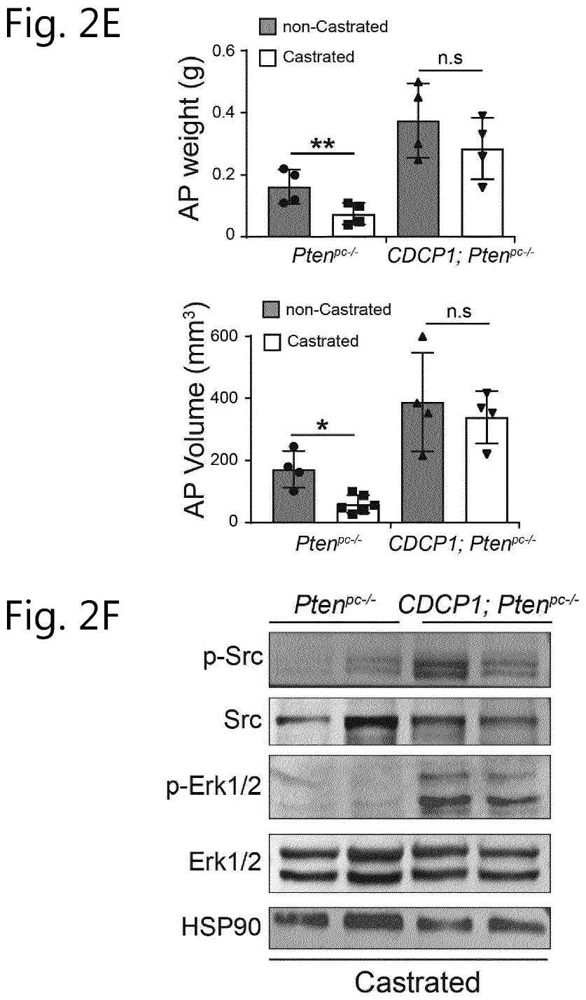

We checked whether overexpression of CDCP1 in Pten-deficient tumours would exacerbate prostate tumorigenesis. Note that, complete Pten loss results in high grade prostatic intraepithelial neoplastic lesions (HGPIN) which progresses to focally invasive adenocarcinoma without metastasis (Alimonti et al., 2010. Nat Genet 42: 454-458; Chen, et al., 2005. Nature 436: 725-730; Trotman et al., 2006. Cell 128:141-156). Strikingly, by the age of 16 weeks CDCP1; Pten^{pc-/-} developed focally invasive adenocarcinoma, that progressed to highly aggressive carcinoma at the age of 36 weeks, a phenotype that was never observed in Pten^{pc-/-} tumours (FIG. 2A). Moreover, the percentage of Ki-67 positive cells was significantly increased in CDCP1; Pten^{pc-/-} mice when compared to their counterpart mice (FIG. 2B). Importantly, pathological analysis of CDCP1; Ptenpc^{-/-} mice revealed metastatic spread of epithelial tumour nodules positive for Pan-Cytokeratin (PanK), CDCP1 and androgen receptor (AR) to draining lumbar lymph nodes in 50 percent of the cases (n=4/8) analyzed and lung metastasis in 12 percent of cases (n=1/9) (data not shown). The histological features of these metastases resembled to those of the primary prostate tumours (data not shown). By contrast, Pten^{pc-/-} mice did not develop metastasis, as previously reported (Chen, et al., 2005. Nature 436: 725-730; Ding et al., 2011. Nature 470: 269-273; Qin et al., 2013. Nature 493: 236-240). CDCP1; Pten^{-/-} MEFs have also an increased proliferative and migratory capacity when compared to Pten^{-/-} cells (data not shown) Additionally, Kaplan-Meier cumulative survival analysis showed that CDCP1; Pten^{pc-/-} mice had to be euthanized or died due to extensive tumour burden at the age of 60-80 weeks (FIG. 2C). Of note, none of the Pten^{pc-/-} mice died at the same age, demonstrating that overexpression of CDCP1 in combination with Pten-deficiency has a profound effect on the survival of the transgenic mice. Moreover, the percentage of Ki-67 positive cells was significantly higher in CDCP1; Pten^{pc-/-} mice when compared to their counterpart mice (data not shown). This was also confirmed in human prostate cancer, where the levels of PTEN negatively correlated with CDCP1 overexpression levels. Moreover, in patients affected by prostate tumors with decreased levels of PTEN and increased level of CDCP1 we observed a short disease free survival (DFS) and overall survival (OS), thereby validating the relevance of the findings in the mouse model (data not shown).

Western Blot (WB) analysis in tumors reveled that CDCP1 overexpression in Pten^{pc-/-} tumors promote the activation of SRC and the following upregulation of p-ERK1/2, while p-Akt was not changed compared to Pten^{pc-/-} tumors. Thus in these tumors while Pten loss drive the activation of p-AKT, CDCP1 promoted the upregulation of the MAPK pathway (FIG. 2D). Since activated Src is known to regulate c-Myc levels (Furstoss et al., 2002. EMBO J 21: 514-524; Jain et al., 2015. Cancer Res 75: 4863-4875; Barone and Courtneidge, 1995. Nature 378: 509-512), we reasoned that CDCP1 overexpression could drive c-Myc overexpression through Src. Indeed, CDCP1 overexpressing tumors showed increased levels of c-Myc expression (data not shown). Furthermore, IHC analysis revealed high levels of c-Myc and pErkl/2 in CDCP1; Pten^{pc-/-} tumors compared to Pten^{pc-/-} tumors (data not shown).

We next checked whether CDCP1 overexpression in Pten null tumours could also promote resistant to androgen deprivation therapy (ADT). To this end, we performed surgical castration in parallel in both Pten^{pc-/-} and CDCP1; Pten^{pc-/-} mice. Macroscopic analysis revealed that while Pten^{pc-/-} tumours responded to castration (Lunardi et al., 2013. Nat Genet 45: 747-755), CDCP1; Pten^{pc-/-} tumours did not, as shown by quantification of tumour weight, volume, histopathological analysis and IHC for Ki-67 (FIG. 2E). Resistance to castration in CDCP1; Pten^{pc-/-} tumours was associated to increased levels of p-SRC/MAPK when compared to Pten^{pc-/-} tumours thus explaining the emergence of CRPC in this genetic background (FIG. 2 F). These data were validated in an additional allograft model of prostate cancer where CDCP1 was overexpressed in TRAMPC1 mouse prostate epithelial cells. In this model overexpression of CDCP1 significantly increased the levels of pSRC and pERK and accelerated the emergence of CRPC when compared to control tumors. Moreover CDCP1 overexpression, shortened the survival of tumor bearing mice (data not shown).

Overexpression of CDCP1 Overcomes Pten-Loss Induced Cellular Senescence and Leads to CRPC by Activating the Src/MAPK/Myc Pathway

Previous evidence demonstrated that Pten^{pc-/-} mice develop indolent tumours characterized by a senescence response that acts as an intrinsic barrier that constrain prostate cancer progression (Chen, et al., 2005. Nature 436: 725-730; Alimonti et al., 2010. J Clin Invest 120: 681-693). Since CDCP1 accelerates tumour progression in Pten^{pc-/-} mice, we tested whether CDCP1 overexpression in this genetic background could promote senescence evasion both in vitro and in vivo leading to mCRPC. Prostate sections of the various genotypes (WT, CDCP1, Pten^{pc-/-} and CDCP1; Pten^{pc-/-}) were analyzed for senescence response by performing SA-ß-gal and p-HP1γ staining an additional marker of senescence in vivo (Di Mitri et al., 2014. Nature 515: 134-137). While a strong cellular senescence response was observed in the PtenPe^{-/-} tumours, CDCP1; Pten^{pc-/-} tumour sections stained negative for both SA-ß-gal and p-HP1γ and positive for Cyclin D1 (data not shown) demonstrating that CDCP1 bypasses senescence driven by Pten-loss.

CDCP1; Pten^{-/-} MEFs also stained negative for SA-ß-gal and exhibited increased cell proliferation with an elongated phenotype when compared to Pten^{-/-} MEFs (data not shown).

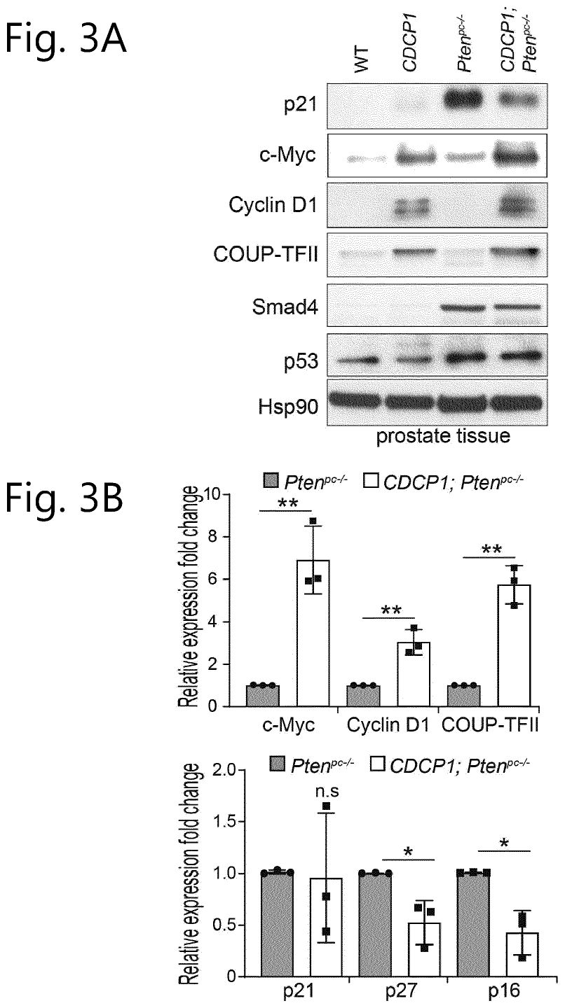

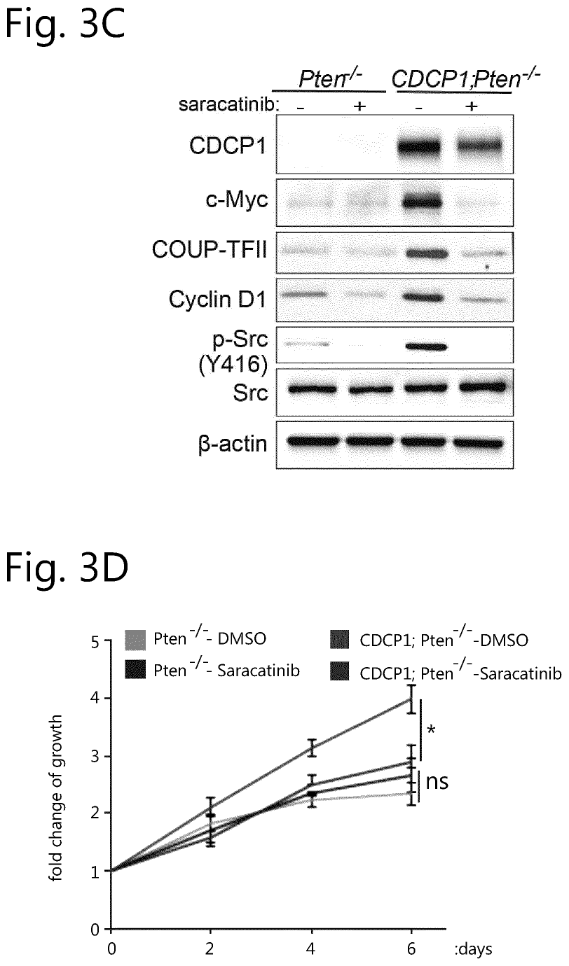

Two recent independent reports showed that upregulation of the TGF-β/Smad4 pathway triggered by PTEN loss constrain prostate cancer progression by blocking Cyclin D1 transcription (Ding et al., 2011. Nature 470: 269-273; Qin et al., 2013. Nature 493: 236-240). Of interest, overexpression of COUP-TFII which inhibits Smad4-dependent transcription, promotes senescence evasion by releasing cyclin D1 expression in PTEN null cells (Ding et al., 2011. Nature 470: 269-273; Qin et al., 2013. Nature 493: 236-240). Thus, we compared the status of several components involved in these pathways such as p53, p21, Smad4, cyclin D1 and COUP-TFII in Ptenpc^{-/-} and CDCP1; Ptenpc^{-/-} tumour samples. While our analysis showed that Smad4 and p53 expression did not change in CDCP1; Pten-null MEFs and tumours compared to control groups, p21, Cyclin D1 and COUP-TFII levels were significantly altered (FIG. 3 A, B). These data suggest that CDCP1 allows PTEN-null benign tumours to acquire metastatic potential through the evasion of the TGF-6-induced senescence barrier by increasing the level of COUP-TFII. We next tried to understand how CDCP1 controlled the expression of COUP-TFII. Analysis of the COUP-TFII promoter exhibited the presence of multiple MYC-binding sites (data not shown). Since CDCP1 controls Src activation which in turn regulates c-Myc levels (Furstoss et al., 2002. EMBO J 21: 514-524; Jain et al., 2015. Cancer Res 75: 4863-4875; Barone and Courtneidge, 1995. Nature 378: 509-512), we checked the levels of MYC in both CDCP1 and CDCP1; Pten null MEFs and tumours and we found a significant induction of c-Myc at both mRNA and protein levels (FIG. 3A, B). Interestingly, c-Myc, COUP-TFII and cyclin D1 mRNA and protein levels were significantly reduced in CDCP1; Pten^{-/-} MEFs upon treatment with saracatinib, an inhibitor of Src20 (FIG. 3C). Of note, treatment with saracatinib led to a profound arrest in the proliferation and reactivation of senescence in CDCP1; Pten^{-/-} MEFs (FIG. 3D). Silencing c-Myc in CDCP1; Pten^{-/-} MEFs phenocopied the results of the Src inhibitor thereby validating our findings that Myc regulates COUP-TFII levels in CDCP1; Pten^{pc-/-} tumours (data not shown). Chromatin immunoprecipitation (ChIP) assays confirmed that c-Myc specifically binds to the promoters of cyclin D1 and COUP-TFII in CDCP1; Pten^{-/-} MEFs when compared to Pten^{-/-} MEFs. Additional ChIP analysis showed increased binding of c-Myc on Cyclin D1 promoter and reduced Smad4 binding affinity to the promoter of Cyclin D1 in CDCP1; Pten^{-/-} MEFs compared to Pten^{-/-} (data not shown). Taken together these data demonstrate that in CDCP1; Pten^{pc-/-} tumours, increased levels of c-Myc promote activation of COUPTF-II that prevents Smad4 to bind the promoter of Cyclin D1. As result, these tumours by-pass senescence and progress towards a metastatic and CRPC phenotype.

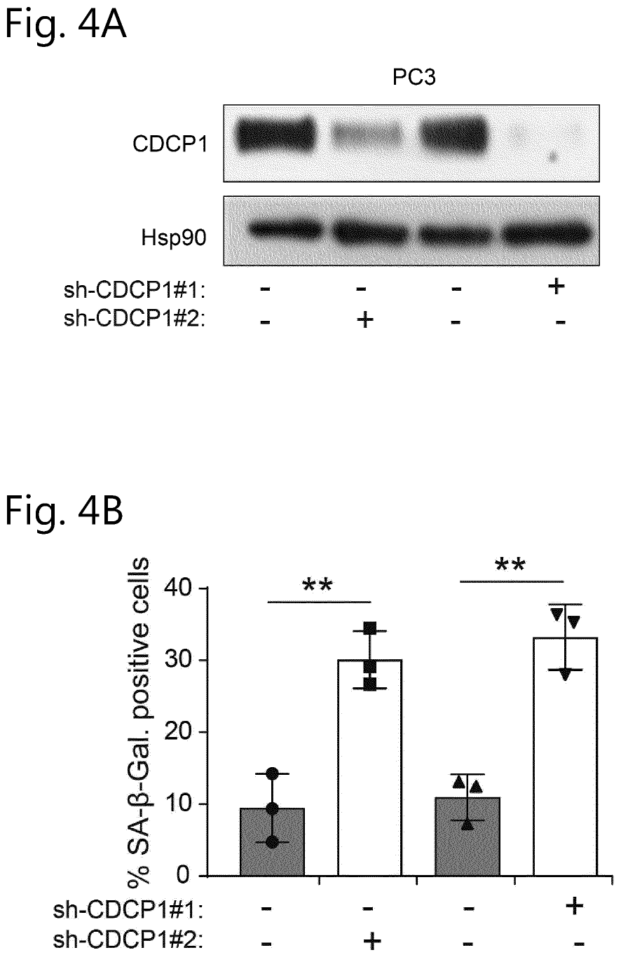

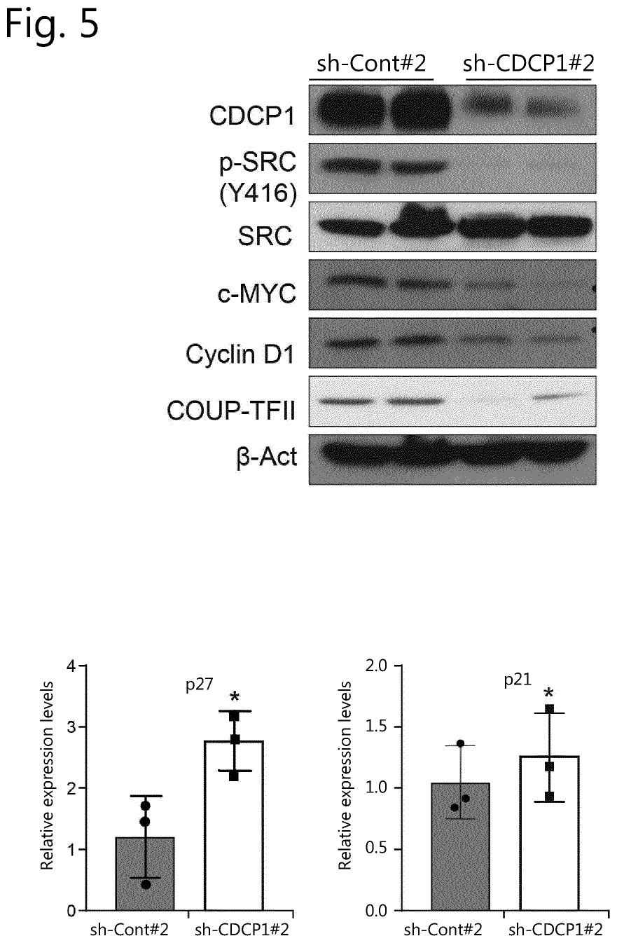

We next reasoned that inhibition of CDCP1 could drive senescence activation in prostate cancer harboring elevated levels of CDCP1. We therefore depleted CDCP1 in PC3, a PTEN; TP53-deficient human prostate cancer cell line by using two independent shRNAs (FIG. 4A). Remarkably, silencing of CDCP1 inhibited 3D proliferation and migration of PC3 cells (data not shown) and promoted senescence as assessed by SA-ß-gal staining positivity (FIG. 4B). In line with this evidence, we injected PC3 cells expressing sh-CDCP1 and its scrambled control subcutaneously in vivo. Both tumour size and weight were dramatically reduced in sh-CDCP1 tumours compared to the control (data not shown). CDCP1 depleted PC3-tumours also showed significant decrease in c-Myc COUP-TFII, and Cyclin D1 expression in parallel with the reduction of SRC phosphorylation. Additionally, two markers of senescence such as p21 and p27 were robustly upregulated in tumours lacking CDCP1 (Lin et al., 2010. Nature 464: 374-379) (FIG. 5). Taken together these data demonstrate that CDCP1 inhibition promote senescence by suppressing c-MYC levels in prostate cancer cells. These results were validated also in LNCaP-abl, an androgen insensitive cell line derived from LNCaP that shown higher level of CDCP1 compared with LNCaP parental (data not shown). Down-regulation of CDCP1 in this cell line decreased proliferation and increased senescence.

Androgen Deprivation Therapy (ADT) Induces CDCP1 Expression and CDCP1 Inhibition Improves the Efficacy of ADTs

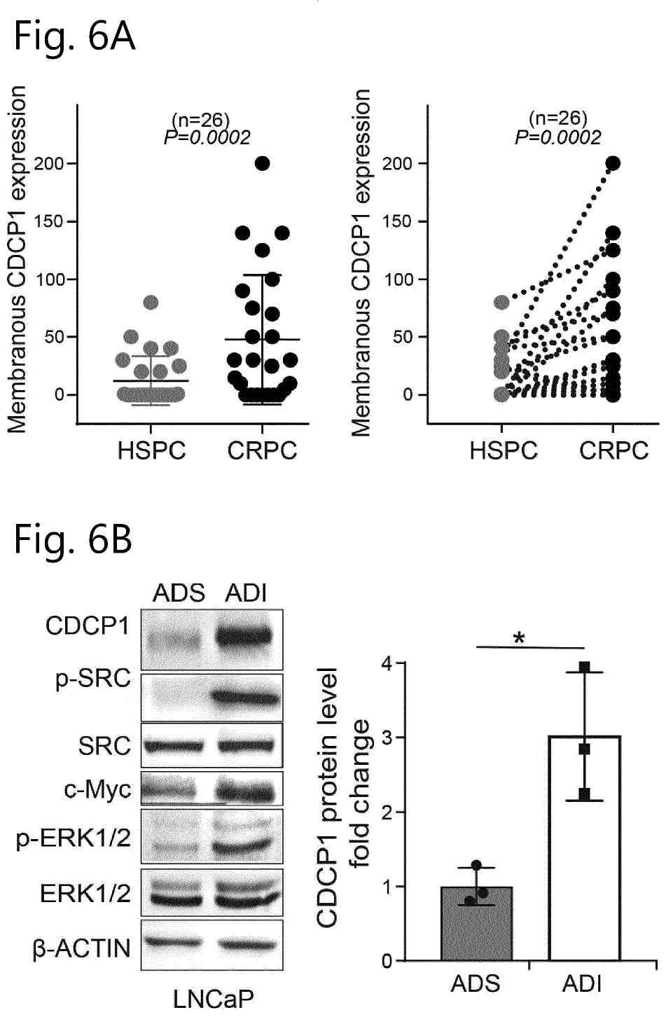

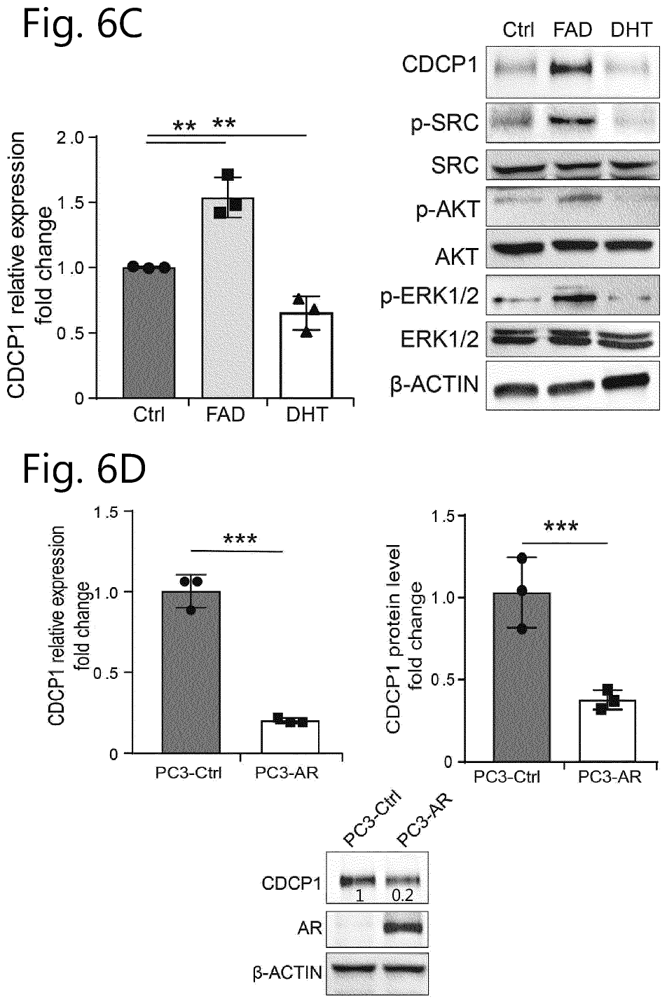

Since CDCP1 is overexpressed in tumors samples from mCRPC patients we checked whether the overexpression of CDCP1 in cancer was triggered by ADT. We therefore extended our examination by checking CDCP1 levels in consecutive sections from a longitudinal study involving patients treated with different lines of ADT. IHC analysis demonstrated that CDCP1 levels significantly increased as patients progressed from HSPC (median HS 0; Interquartile range [IQR] 0.0 to 22.5) to mCRPC (30; 0.0 to 82.5) (FIG. 6A). To further validate these data in vitro we cultured the androgen-sensitive LNCaP cell line under full androgen deprivation (FAD) condition for more than 40 days and we waited until these cells developed resistance. Notably, CDCP1 expression and protein levels increased in cells that become resistant to enzalutamide (ADI) when compared to enzalutamide sensitive cells (ADS). This upregulation was associated to a concomitant activation of p-SRC, c-Myc and p-ERK1/2 and to evasion of senescence driven by enzalutamide treatment (FIG. 6B).

These results promoted us to investigate whether AR signaling regulates the expression of CDCP1. Indeed, FAD treatment enhanced CDCP1 levels, while dihydrotestosteron (DHT) stimulation reduced its expression and protein levels in LNCaP ADS cells (FIG. 6C). In addition, overexpression of AR reduced the expression of CDCP1 in AR negative prostate cancer cell line PC3 (FIG. 6D). This phenotype was abolished when a mutated form of AR with a deletion of the DNA binding domain was overexpressed in the same cells (data not shown). Finally, ChIP-quantitative PCR (ChIP-qPCR) analyses indicated that AR was directly recruited to the CDCP1 proximal promoter and presumably inhibited CDCP1 transcription.

We next investigated whether loss of PTEN was needed for the upregulation of CDCP1 in cells kept in FAD. Indeed, CDCP1 levels increased in PTEN null LNCaP cell but not in the PTEN wild type LAPC4 and VCaP cell lines kept in FAD (data not shown). In line with these findings, we found that in the ADT insensitive cell lines PC3 and 22RV1, FAD did not upregulate CDCP1 levels. Interestingly, inhibition of PI3K in LNCaP cells, but not in 22RV1 cells, promoted a downregulation of CDCP1 in cells kept in FAD. This was associated with a concomitant upregulation of AR levels in the same cells. These data are coherent with previous findings demonstrating that PTEN loss leads to reciprocal feedback inhibition of AR activity (Carver et al., 2011. Cancer Cell 19: 575-586). Thus, inhibition of PI3K leads to increased AR levels that promote the following down-regulation of CDCP1.

### CDCP1-Targeting Improves the Efficacy of ADTs

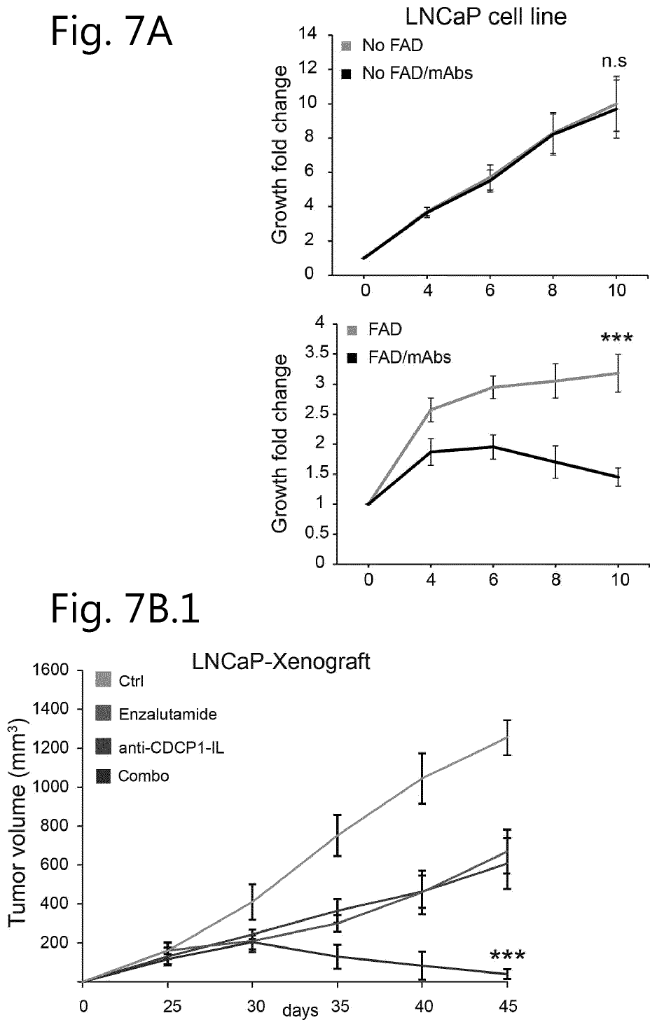

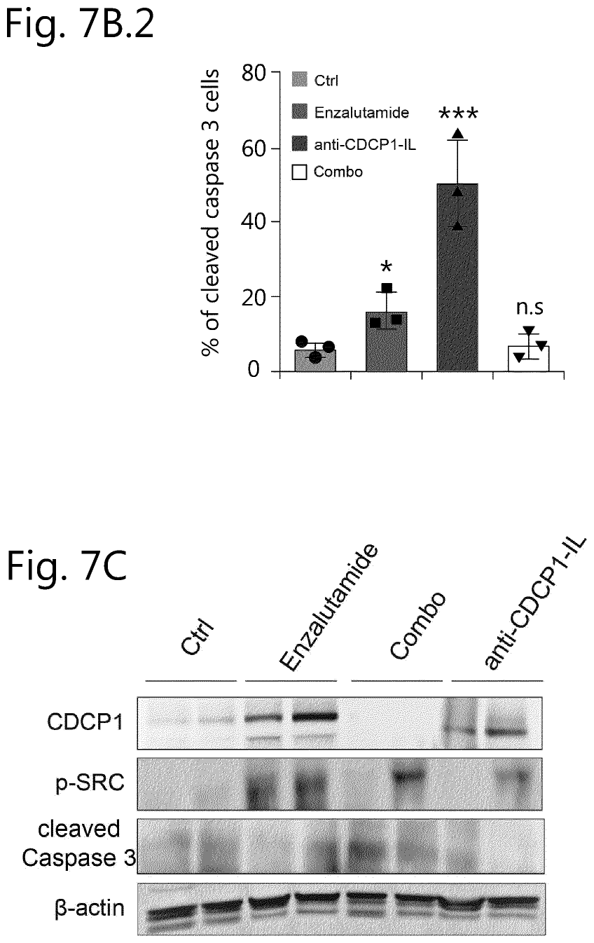

These findings were further validated in LNCaP-abl cells, an androgen insensitive cell line. In these cells, we found that CDCP1 levels were upregulated when compared to LNCap parental cells. Of note, ChIP-qPCR showed that AR was incapable to bind the CDCP1 promoter in these cells. Interestingly, AR activity in patients affected by CRPC anti-correlated with CDCP1 expression. Given the fact that CDCP1 expression is elevated by androgen deprivation, we postulated that targeting CDCP1 could serve as a powerful tool to treat prostate cancers treated with enzalutamide. In support of this notion, treatment with humanized monoclonal antibody targeting CDCP1 (Kollmorgen et al., 2013. Mol Oncol 7: 1142-1151) in cells treated with enzalutamide strongly affected proliferation-inducing senescence and cell death (FIG. 7A) when compared to enzalutamide untreated cells. Note that treatment with the CDCP1-mAbs strongly decreased the levels of CDCP1 in enzalutamide-treated LNCaP cells (FIG. 7C). These data were also validated in LNCaP-abl that overexpressed CDCP1 when compared to their parental cells (data not shown). Since senescence works as a double edge sword that may stimulate cancer cell growth in certain conditions, we decided to develop a novel anti-CDCP1 dual steal immunoliposome carrying doxorubicin (anti-CDCP1 ILs) to eliminate the CDCP1 overexpressing resistant cells. Enzalutamide treatment in combination with anti-CDCP1 ILs induced a strong apoptotic response and blocked the emergence of CDCP1+ ADI cells. To validate these data in vivo, LNCaP cells were injected subcutaneously into SCID-mice and once tumors were established, mice were treated with Enzalutamide (10 mg/kg) with or without anti-CDCP1 ILs for one week. Importantly, while Enzalutamide and anti-CDCP1 ILs alone showed minor effect on tumor growth, combination of Enzalutamide and anti-CDCP1 significantly blocked tumour growth and progression by inducing apoptosis (FIG. 7B). Note that Western blot analysis showed significant increased levels of CDCP1 upon Enzalutamide treatment, which was abolished upon combination treatment (FIG. 7C). Together these data suggest that CDCP1 targeting agents are effective when used in combination with ADTs.

In summary, our study provides evidence that CDCP1 overexpression initiates tumorigenesis in several transgenic models, and cooperates with additional oncogenic events such as loss of tumour suppressor gene Pten to drive tumour progression. Further, we demonstrate that overexpression of CDCP1 in Pten-null prostate tumours bypasses Pten-null induced senescence barrier thereby allowing tumour-progression and metastatic castration resistant prostate cancer trough the activation of the MAPK pathway. Of note we demonstrate that ADT enhances the levels of CDCP1 in cancer cells leading to CRPC. Inactivation of CDCP1 in prostate cancer cells by means of either a CDCP1_shRNA or a therapeutic antibody abrogated the c-Myc-Src axis thereby eliciting cellular senescence. Finally we have reported for the first time that an anti-CDCP1 immunoliposome can enhance the efficacy of enzalutamide treatment opening the possibility that this combination of compounds may be used in the clinic to prevent the occurrence of enzalutamide resistant CDCP1+ tumor cells.

Given that CDCP1 is a cell surface protein which can be targeted by various monoclonal antibodies and small molecule inhibitors (Nakashima et al., 2017. Cancer Sci 108: 1049-1057; Kollmorgen et al., 2013. Mol Oncol 7: 1142-1151; Siva et al., 2008. Cancer Res 68: 3759-3766), our study also warrants a novel therapeutic approach to target castration-resistant and metastatic prostate tumours with elevated CDCP1 levels. Indeed our findings that mutated AR does not efficiently bind and suppress CDCP1 levels suggest that tumors harboring AR mutations or splicing variants possess higher levels of CDCP1 and may benefit of treatments employing either a CDCP1 monoclonal antibody or an anti-CDCP1 ILs.

Thus, targeting CDCP1 could also represent an alternative therapeutic strategy to target CRPC patients after the failure of ADT.

### Example 2

To further assess the clinical relevance of CDCP1 in human prostate cancer (PCa), the number of tumor microarrays (TMAs) was expanded to a total of 990 cases spanning from benign, primary and metastatic PCa. Immunohistochemical (IHC) analysis showed that, while a large portion of prostate tumors analyzed did not express CDCP1, a subset (48%) of CRPC and metastatic tumor samples expressed high level of CDCP1 (data not shown)). In line with these findings, analysis of consecutive tumor samples from a longitudinal study revealed that, in PCa patients, CDCP1 was upregulated during the transition from hormone-sensitive to CRPC (data not shown). Intriguingly, high levels of CDCP1 correlated with decreased levels of PTEN in both primary, CRPC and metastatic prostate tumor samples (Table 3, 4).

TABLE 3
Primary tumors

| | CDCP1-negative | CDCP1-positive |
|---|---|---|
| PTEN-normal | 259 | 39 |
| PTEN-low | 66 | 22 |

The chi-square statistic is 7.246. The p-value is .007106.
The result is significant at p < .05.

TABLE 4
CRPC/Metastasis

| | CDCP1-negative | CDCP1-positive |
|---|---|---|
| PTEN-normal | 40 | 20 |
| PTEN-negative | 23 | 33 |

The chi-square statistic is 7.6471. The p-value is .005686.
The result is significant at p < .05.

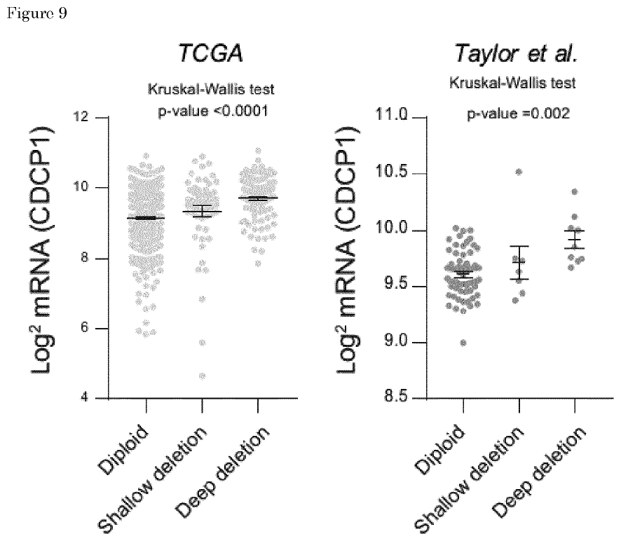

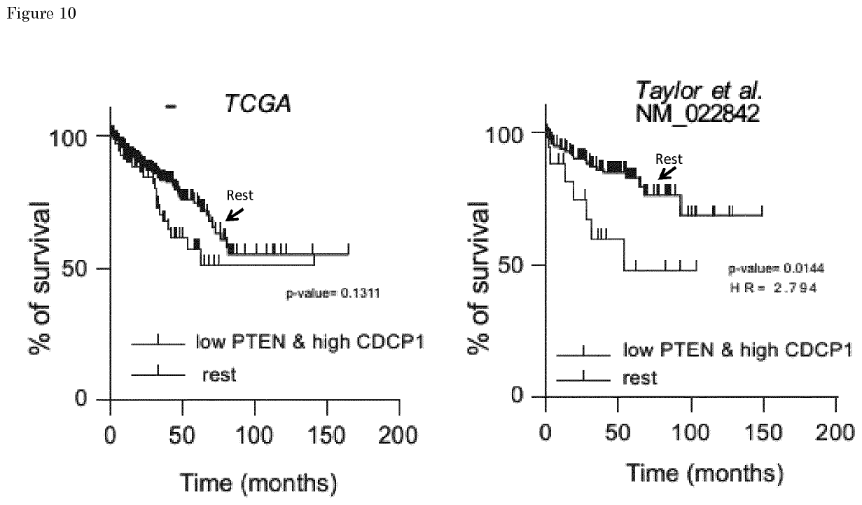

The frequency of tumors displaying a low level of PTEN and high levels of CDCP1 increased in CRPCs and metastatic tumors when compared to primary tumors, thereby validating the clinical relevance of this anti-correlation. Additionally, bioinformatics analysis evaluating different datasets confirmed the existence of an anti-correlation between PTEN and CDCP1 mRNA levels (data not shown). Elevated levels of CDCP1 expression were also significantly associated with PTEN genetic deletions and low CDCP1 promoter methylation in different independent data sets of PCa (FIG. 9). Although patients affected by prostate tumors harboring high level of CDCP1 had a similar disease-free survival (DFS) than patients with low CDCP1, patients with tumors expressing low levels of PTEN and increased level of CDCP1, had a significantly shorter DFS than patients of the other categories (FIG. 10). Taken together, these data validate the clinical relevance of CDCP1 and suggest that CDCP1 could cooperate with the loss of PTEN to promote highly aggressive prostate cancer.

1. A method of treating a patient suffering from castrate-resistant prostate cancer, comprising
providing said patient with a downmodulator of CUB domain-containing protein 1 (CDCP1).

2. The method according to claim 1, wherein the downmodulator of CDCP1 use comprises an antibody that recognizes an extracellular epitope of CDCP1.

3. The method according to claim 1, wherein the patient is provided with the downmodulator in combination with anti-androgen therapy.

4. The method according to claim 3, wherein the anti-androgen therapy is or comprises an androgen receptor antagonist.

5. The method according to claim 3, wherein the anti-androgen therapy is or comprises an anti-androgen selected from enzalutamide, abiraterone, bicalutamide, and nilutamide.

6. The method according to claim 1, wherein the provision of the downmodulator to the patient is combined with a senolytic compound, a genotoxic agent, or a combination of a senolytic compound and a genotoxic agent.

7. The method according to claim 6, wherein the senolytic compound is selected from rapamycin, ABT263, FOXO4-DRI, a CXCR4 inhibitor, a CXCR4 antagonist, a combination of a CXCR4 inhibitor and a CXCR4 antagonist, and dasatinib.

8. A pharmaceutical composition, comprising a downmodulator of CDCP1 and one or more of: an androgen receptor antagonist, a senolytic compound, and/or a genotoxic agent, or a combination of a senolytic compound and a genotoxic agent.

9. The pharmaceutical composition according to claim 8, comprising a pharmaceutical preparation comprising the downmodulator of CDCP1, and a pharmaceutical preparation comprising the androgen receptor antagonist, the senolytic compound, the genotoxic agent, or the combination of the senolytic compound and the genotoxic agent.

10. The pharmaceutical composition according to claim 8, wherein the downmodulator of CDCP1 is present in liposomes.

11. A method according to claim 1 of treating a patient suffering from castrate-resistant prostate cancer, comprising providing the pharmaceutical preparation according to claim 8.

12. A method of selecting a patient with prostate cancer eligible for treatment with a combination of a downmodulator of CDCP1 and a senolytic compound, comprising
determining a level of testosterone in a bodily fluid of the patient;
identifying a patient of which the level of testosterone is below 50 ng/dL;
determining whether the prostate cancer is progressing in the identified patient; and
selecting a patient in which the testosterone level is below 50 ng/dL and in which prostate cancer is progressing as a patient who is eligible for treatment with a combination of a downmodulator of CDCP1 and a senolytic compound.

13. The method of claim 12, wherein progression of prostate cancer in the identified patient is determined by a continuous rise in serum prostate-specific antigen (PSA) levels, the appearance of new metastases in said patient, or a combination thereof.

14. A method according to claim 1 of treating the patient suffering from castrate-resistant prostate cancer with a combination of the downmodulator of CDCP1 and a senolytic compound, the method further comprising:
determining a level of testosterone in a bodily fluid of the patient;
identifying a patient of which the level of testosterone is below 50 ng/dL;
determining whether the prostate cancer is progressing in the identified patient; and
treating a patient in which the testosterone level is below 50 ng/dL and in which prostate cancer is progressing with a combination of a downmodulator of CDCP1 and a senolytic compound, a combination of a downmodulator of CDCP1 and a genotoxic agent, or with a combination of a downmodulator of CDCP1, a senolytic compound and a genotoxic agent.

15. A method of treating a patient suffering from prostate cancer with a combination of an antibody-drug conjugate and an androgen receptor antagonist, whereby the antibody recognizes an extracellular epitope of CDCP1, whereby the drug conjugate is a chemotherapeutic drug, a toxic compound or a radioactive compound.

16. A method of treating a patient with castrate-resistant prostate cancer, comprising
identifying a patient who suffers from castrate-resistant prostate cancer; and
treating said identified patient with a combination of a downmodulator of CDCP1 and a senolytic compound, with a combination of a downmodulator of CDCP1 and a genotoxic agent, or with a combination of a downmodulator of CDCP1, a senolytic compound and a genotoxic agent.

17. The pharmaceutical composition according to claim 10, wherein said liposomes further comprise an anthracycline such as doxorubicin.

18. The method according to claim 14, wherein the downmodulator is administered prior to the administration of the senolytic compound, the genotoxic agent, or combination thereof.

19. The method of claim 15, whereby the anti-androgen is enzalutamide.

20. The method of claim 16, wherein the downmodulator is administered prior to the administration of the senolytic compound, the genotoxic agent, or combination thereof.


SEQUENCE LISTING (116 sequences)

SEQ ID NO: 1 - protein, 46 aa, Artificial Sequence
  FOXO4-DRI
LTLRKEPASEIAQSILEAYSQNGWANRRSGGKRPPPRRRQRRKKRG

SEQ ID NO: 2 - DNA, 19 bp, Artificial Sequence
  V3THS_329377 RNAi
tgagggtaggcaacaacga

SEQ ID NO: 3 - DNA, 19 bp, Artificial Sequence
  V2THS_191307 RNAi
tctttctccagacttgatg

SEQ ID NO: 4 - DNA, 25 bp, Artificial Sequence
  primer
aaaagttcccctgatgatgatttgt

SEQ ID NO: 5 - DNA, 28 bp, Artificial Sequence
  primer
tgtttttgaccaattaaagtaggctgtg

SEQ ID NO: 6 - DNA, 25 bp, Artificial Sequence
  primer
ttctcttgagcactgtttcacaggc

SEQ ID NO: 7 - DNA, 21 bp, Artificial Sequence
  primer
tgatggacatgttcagggatc

SEQ ID NO: 8 - DNA, 17 bp, Artificial Sequence
  primer
gccaccagtctgcatga

SEQ ID NO: 9 - DNA, 20 bp, Artificial Sequence
  primer
caagggagaagagagtgcgg

SEQ ID NO: 10 - DNA, 20 bp, Artificial Sequence
  primer
cccaacaatggggatgtaag

SEQ ID NO: 11 - DNA, 31 bp, Artificial Sequence
  primer
gatatccaccatggccggcctgaactgcggg

SEQ ID NO: 12 - DNA, 27 bp, Artificial Sequence
  primer
actagttcaatggtgatggtgatgatg

SEQ ID NO: 13 - DNA, 20 bp, Artificial Sequence
  primer
cgcaggttcttggtcactgt

SEQ ID NO: 14 - DNA, 20 bp, Artificial Sequence
  primer
tgttcacgaaagccagagcg

SEQ ID NO: 15 - DNA, 20 bp, Artificial Sequence
  primer
gcaaaacaaaagggccaaca

SEQ ID NO: 16 - DNA, 18 bp, Artificial Sequence
  primer
gggcgtctgctccacagt

SEQ ID NO: 17 - DNA, 21 bp, Artificial Sequence
  primer
aggtcggtgtgaacggatttg

SEQ ID NO: 18 - DNA, 21 bp, Artificial Sequence
  primer
tgtagaccatgtagttgaggt

SEQ ID NO: 19 - DNA, 23 bp, Artificial Sequence
  primer
tggattcgacttagacttgacct

SEQ ID NO: 20 - DNA, 19 bp, Artificial Sequence
  primer
gcggtgtcataatgtctct

SEQ ID NO: 21 - DNA, 19 bp, Artificial Sequence
  primer
accgcagctaggaataatg

SEQ ID NO: 22 - DNA, 19 bp, Artificial Sequence
  primer
gcctcagttccgaaaacca

SEQ ID NO: 23 - DNA, 20 bp, Artificial Sequence
  primer
tcaactgccactcgtacctg

SEQ ID NO: 24 - DNA, 18 bp, Artificial Sequence
  primer
catgatgttgttaggctg

SEQ ID NO: 25 - DNA, 19 bp, Artificial Sequence
  primer
gcgtaccctgacaccaatc

SEQ ID NO: 26 - DNA, 21 bp, Artificial Sequence
  primer
ctcctcttcgcacttctgctc

SEQ ID NO: 27 - DNA, 19 bp, Artificial Sequence
  primer
ctggaccagggagtggagt

SEQ ID NO: 28 - DNA, 19 bp, Artificial Sequence
  primer
acgtagtagtcggttctca

SEQ ID NO: 29 - DNA, 19 bp, Artificial Sequence
  primer
tgtccgtcagaacccatgc

SEQ ID NO: 30 - DNA, 21 bp, Artificial Sequence
  primer
aaagtcgaagttccatcgctc

SEQ ID NO: 31 - DNA, 21 bp, Artificial Sequence
  primer
taattggggctccggctaact

SEQ ID NO: 32 - DNA, 21 bp, Artificial Sequence
  primer
tgcaggtcgcttccttattcc

SEQ ID NO: 33 - DNA, 20 bp, Artificial Sequence
  primer
aatcccatcaccatcttcca

SEQ ID NO: 34 - DNA, 20 bp, Artificial Sequence
  primer
tggactccacgacgtactca

SEQ ID NO: 35 - DNA, 20 bp, Artificial Sequence
  primer
cggaactcttgtgcgtaagg

SEQ ID NO: 36 - DNA, 20 bp, Artificial Sequence
  primer
ctcagccaaggttgtgaggt

SEQ ID NO: 37 - DNA, 20 bp, Artificial Sequence
  primer
tggttccaccccagaaatgt

SEQ ID NO: 38 - DNA, 21 bp, Artificial Sequence
  primer
gatgatgcacagacgttttat

SEQ ID NO: 39 - DNA, 18 bp, Artificial Sequence
  primer
tcgtgggcgaggggctgt

SEQ ID NO: 40 - DNA, 18 bp, Artificial Sequence
  primer
catccgaacgccttcatc

SEQ ID NO: 41 - DNA, 18 bp, Artificial Sequence
  primer
tactctggaagttcatgg

SEQ ID NO: 42 - DNA, 18 bp, Artificial Sequence
  primer
gtcatccactattccttg

SEQ ID NO: 43 - DNA, 18 bp, Artificial Sequence
  primer
ctgcccattgcctaaaga

SEQ ID NO: 44 - DNA, 15 bp, Artificial Sequence
  primer
gtagagcgggtgtgg

SEQ ID NO: 45 - DNA, 15 bp, Artificial Sequence
  primer
ggaaatgaccaggcc

SEQ ID NO: 46 - DNA, 18 bp, Artificial Sequence
  primer
ccagcttctgctcagtgc

SEQ ID NO: 47 - DNA, 22 bp, Artificial Sequence
  primer
ccgcttagtccccattctaaag

SEQ ID NO: 48 - DNA, 22 bp, Artificial Sequence
  primer
ggcatctccattcttaatccag

SEQ ID NO: 49 - DNA, 21 bp, Artificial Sequence
  primer
gtgcggggacaagtcgagcgg

SEQ ID NO: 50 - DNA, 21 bp, Artificial Sequence
  primer
gcggtggtgctggtcgatggg

SEQ ID NO: 51 - protein, 120 aa, Artificial Sequence
  heavy chain
EVQLVESGGDLVKPGGSLKLSCAASGFTFNSYGMSWVRQTPDKRLEWVATISSGGSYTYYPDSVKGRFTISRDNAKNTLYLQMSSLKSEDTAMYYCARHPDYDGVWFAYWGQGTLVTVSA

SEQ ID NO: 52 - protein, 5 aa, Artificial Sequence
  CDR1
SYGMS

SEQ ID NO: 53 - protein, 16 aa, Artificial Sequence
  CDR2
TISSGGSYTYYPDSVK

SEQ ID NO: 54 - protein, 10 aa, Artificial Sequence
  CDR3
HPDYDGVWFA

SEQ ID NO: 55 - protein, 7 aa, Artificial Sequence
  CDR1
TAGVGVS

SEQ ID NO: 56 - protein, 16 aa, Artificial Sequence
  CDR2
HIYWDDDKRYNPSLKS

SEQ ID NO: 57 - protein, 10 aa, Artificial Sequence
  CDR3
STSVEEAFDV

SEQ ID NO: 58 - protein, 7 aa, Artificial Sequence
  CDR1
TAGMGVS

SEQ ID NO: 59 - protein, 9 aa, Artificial Sequence
  CDR3
SDSFGDFDY

SEQ ID NO: 60 - protein, 5 aa, Artificial Sequence
  CDR1
NFGLH

SEQ ID NO: 61 - protein, 16 aa, Artificial Sequence
  CDR2
VIWSGGVTDYNAAFIS

SEQ ID NO: 62 - protein, 10 aa, Artificial Sequence
  CDR3
NYDHDYSMDY

SEQ ID NO: 63 - protein, 120 aa, Artificial Sequence
  heavy chain
QVTLKESGPGILQPSQTLSLTCSFSGFSLSTAGVGVSWIRQPSGKGLEWLAHIYWDDDKRYNPSLKSRLTISKDTSRNQVFLKITSVDSADTATYFCSRSTSVEEAFDVWGAGTTVTVSS

SEQ ID NO: 64 - protein, 10 aa, Artificial Sequence
  CDR1
GFSLSTAGVG

SEQ ID NO: 65 - protein, 16 aa, Artificial Sequence
  CDR2
HIYWDDDKRYNPSLKS

SEQ ID NO: 66 - protein, 10 aa, Artificial Sequence
  CDR3
STSVEEAFDV

SEQ ID NO: 67 - protein, 119 aa, Artificial Sequence
  heavy chain
QVTLKESGPGILQSSQTLSLTCSFSGFSLSTAGMGVSWIRQPSGKGLEWLAHIYWDDDKRYNPSLKSRLTVSKGTSRNQVLLKITSVDAADTATYYCARSDSFGDFDYWGQGTTLTVSS

SEQ ID NO: 68 - protein, 118 aa, Artificial Sequence
  heavy chain
QVHLKQSGPGLVQPSQSLSITCTVSGFSLTNFGLHWVRQSPGKGLEWLGVIWSGGVTDYNAAFISRLSISKDNSKSQVFFKMNSLQPNDTAIYYCARNYDHDYSMDYWGQGISVSVSS

SEQ ID NO: 69 - protein, 120 aa, Artificial Sequence
  heavy chain
EVQLVESGGGLVQPGGSLRLSCAASGFTFNSYGMSWVRQAPGKGLEWVATISSGGSYKYYVDSVKGRFTISRDNAKNSLYLQMNSLRAEDTAVYYCARHPDYDGVWFAYWGQGTLVTVSS

SEQ ID NO: 70 - protein, 120 aa, Artificial Sequence
  heavy chain
EVQLVESGGGLVQPGGSLRLSCAASGFTFNSYGMSWVRQAPGKGLEWVATISSGGSYTYYPDSVKGRFTISRDNAKNSLYLQMNSLRAEDTAVYYCARHPDYDGVWFAYWGQGTLVTVSS

SEQ ID NO: 71 - protein, 120 aa, Artificial Sequence
  heavy chain
EVQLVESGGGLVKPGGSLRLSCAASGFTFNSYGMSWVRQAPGKGLEWVSTISSGGSYTYYPDSVKGRFTISRDNAKNSLYLQMNSLRAEDTAVYYCARHPDYDGVWFAYWGQGTLVTVSS

SEQ ID NO: 72 - protein, 120 aa, Artificial Sequence
  heavy chain
EVQLLESGGGLVQPGGSLRLSCAASGFTFNSYGMSWVRQAPGKGLEWVSTISSGGSYTYYPDSVKGRFTISRDNSKNTLYLQMNSLRAEDTAVYYCAKHPDYDGVWFAYWGQGTLVTVSS

SEQ ID NO: 73 - protein, 120 aa, Artificial Sequence
  heavy chain
EVQLLESGGGLVQPGGSLRLSCAASGFTFNSYGMSWVRQAPGKGLEWVSTISSGGSYTYYPDSVKGRFTISRDNSKNTLYLQMNSLRAEDTAVYYCARHPDYDGVWFAYWGQGTLVTVSS

SEQ ID NO: 74 - protein, 120 aa, Artificial Sequence
  heavy chain
EVQLVESGGGLVKPGGSLRLSCAASGFTFNSYGMSWVRQAPGKGLEWVSSISSGGSYIYYADSVKGRFTISRDNAKNSLYLQMNSLRAEDTAVYYCARHPDYDGVWFAYWGQGTLVTVSS

SEQ ID NO: 75 - protein, 120 aa, Artificial Sequence
  heavy chain
EVQLVESGGGLVKPGGSLRLSCAASGFTFNSYGMSWVRQAPGKGLEWVSSISSGGSYTYYADSVKGRFTISRDNAKNSLYLQMNSLRAEDTAVYYCARHPDYDGVWFAYWGQGTLVTVSS

SEQ ID NO: 76 - protein, 120 aa, Artificial Sequence
  heavy chain
EVQLLESGGGLVQPGGSLRLSCAASGFTFNSYAMSWVRQAPGKGLEWVSAISSGGSYTYYADSVKGRFTISRDNSKNTLYLQMNSLRAEDTAVYYCAKHPDYDGVWFAYWGQGTLVTVSS

SEQ ID NO: 77 - protein, 120 aa, Artificial Sequence
  heavy chain
EVQLLESGGGLVQPGGSLRLSCAASGFTFNSYAMSWVRQAPGKGLEWVSTISSGGSYTYYPDSVKGRFTISRDNSKNTLYLQMNSLRAEDTAVYYCAKHPDYDGVWFAYWGQGTLVTVSS

SEQ ID NO: 78 - protein, 120 aa, Artificial Sequence
  heavy chain
EVQLLESGGGLVQPGGSLRLSCAASGFTFNSYGMSWVRQAPGKGLEWVSAISSGGSYTYYPDSVKGRFTISRDNSKNTLYLQMNSLRAEDTAVYYCAKHPDYDGVWFAYWGQGTLVTVSS

SEQ ID NO: 79 - protein, 120 aa, Artificial Sequence
  heavy chain
EVQLVESGGGLVKPGGSLRLSCAASGFTFNSYSMNWVRQAPGKGLEWVSSISSGGSYIYYADSVKGRFTISRDNAKNSLYLQMNSLRAEDTAVYYCARHPDYDGVWFAYWGQGTLVTVSS

SEQ ID NO: 80 - protein, 10 aa, Artificial Sequence
  CDR1
GYTFTSYYMY

SEQ ID NO: 81 - protein, 17 aa, Artificial Sequence
  CDR2
EINPSHGGTNFNEKFKN

SEQ ID NO: 82 - protein, 11 aa, Artificial Sequence
  CDR3
GGNYPYFAMDY

SEQ ID NO: 83 - protein, 106 aa, Artificial Sequence
  light chain
QIVLTQSPAIMSASPGEKVTMTCSVSSSVFYVHWYQQKSGTSPKRWIYDTSKLASGVPARFSGSGSGTSYSLTISSMEAEDAATYYCQQWNSNPPTFGGGTKLEIK

SEQ ID NO: 84 - protein, 10 aa, Artificial Sequence
  CDR1
SVSSSVFYVH

SEQ ID NO: 85 - protein, 7 aa, Artificial Sequence
  CDR2
DTSKLAS

SEQ ID NO: 86 - protein, 9 aa, Artificial Sequence
  CDR3
QQWNSNPPT

SEQ ID NO: 87 - protein, 16 aa, Artificial Sequence
  CDR1
RSSQNIVHSYGNTYLE

SEQ ID NO: 88 - protein, 7 aa, Artificial Sequence
  CDR2
KVSNRFS

SEQ ID NO: 89 - protein, 9 aa, Artificial Sequence
  CDR3
FQGSHVPWT

SEQ ID NO: 90 - protein, 16 aa, Artificial Sequence
  CDR1
KSSQSLLYTNGKTYLI

SEQ ID NO: 91 - protein, 7 aa, Artificial Sequence
  CDR2
LVSKLDS

SEQ ID NO: 92 - protein, 9 aa, Artificial Sequence
  CDR3
LQSTHFPYT

SEQ ID NO: 93 - protein, 16 aa, Artificial Sequence
  CDR1
KSTKSLLNSAGFTYLG

SEQ ID NO: 94 - protein, 7 aa, Artificial Sequence
  CDR2
LVSNRFS

SEQ ID NO: 95 - protein, 9 aa, Artificial Sequence
  CDR3
FQSSYLPLT

SEQ ID NO: 96 - protein, 112 aa, Artificial Sequence
  light chain
DVLMTQTPLSLPVSLGDQASISCRSSQNIVHSYGNTYLEWYLQKPGQSPKLLIYKVSNRFSGVPDRFSGSGSGTDFTLKISRVEAEDLGVYYCFQGSHVPWTFGGGTKLEIK

SEQ ID NO: 97 - protein, 10 aa, Artificial Sequence
  CDR1
RSSQNIVHSY

SEQ ID NO: 98 - protein, 7 aa, Artificial Sequence
  CDR2
KVSNRFS

SEQ ID NO: 99 - protein, 9 aa, Artificial Sequence
  CDR3
FQGSHVPWT

SEQ ID NO: 100 - protein, 112 aa, Artificial Sequence
  light chain
DVVMTQTPLTLSVTIGQPASISCKSSQSLLYTNGKTYLIWLLQRPGQSPKRLIYLVSKLDSGVPDRFSGSGSGTDFTLKISRVEAEDLGVYYCLQSTHFPYTFGGGTKLEIK

SEQ ID NO: 101 - protein, 112 aa, Artificial Sequence
  light chain
DVVLTQTPLSLPVIIGDQASISCKSTKSLLNSAGFTYLGWYLQKPGQSPQLLIYLVSNRFSGVPDRFSGSGSGTDFTLKISRVEAEDLGVYFCFQSSYLPLTFGSGTKLEIK

SEQ ID NO: 102 - protein, 106 aa, Artificial Sequence
  light chain
EIVLTQSPATLSLSPGERATMSCGASSSVFYLHWYQQKPGLAPRLWIYDTSKLASGIPDRFSGSGSGTDFTLTISRLEPEDFAVYYCQQWNSNPPTFGGGTKVEIK

SEQ ID NO: 103 - protein, 106 aa, Artificial Sequence
  light chain
EIVLTQSPATLSLSPGERATMSCSVSSSVFYLHWYQQKPGQAPRLWIYDTSKLASGIPARFSGSGSGTDFTLTISSLEPEDFAVYYCQQWNSNPPTFGGGTKVEIK

SEQ ID NO: 104 - protein, 106 aa, Artificial Sequence
  light chain
EIVLTQSPATLSLSPGERATLSCRASSSVFYLHWYQQKPGQAPRLWIYDTSKLASGIPARFSGSGSGTDFTLTISSLEPEDFAVYYCQQWNSNPPTFGGGTKVEIK

SEQ ID NO: 105 - protein, 106 aa, Artificial Sequence
  light chain
EIVLTQSPATLSLSPGERATMSCRASSSVFYLHWYQQKPGQAPRLWIYDTSKLASGIPARFSGSGSGTDFTLTISSLEPEDFAVYYCQQWNSNPPTFGGGTKVEIK

SEQ ID NO: 106 - protein, 106 aa, Artificial Sequence
  light chain
EIVMTQSPATLSLSPGERATMSCRASSSVFYLHWYQQKPGQAPRLWIYDTSKLASGIPARFSGSGSGTDFTLTISSLQPEDFAVYYCQQWNSNPPTFGGGTKVEIK

SEQ ID NO: 107 - protein, 106 aa, Artificial Sequence
  light chain
EIVLTQSPATLSLSPGERATLSCSVSSSVFYVHWYQQKPGQAPRLLIYDTSKLASGIPARFSGSGSGTDYTLTISSLEPEDFAVYYCQQWNSNPPTFGGGTKVEIK

SEQ ID NO: 108 - protein, 106 aa, Artificial Sequence
  light chain
EIVMTQSPATLSVSPGERATLSCSVSSSVFYVHWYQQKPGQAPRLLIYDTSKLASGIPARFSGSGSGTEFTLTISSLQSEDFAVYYCQQWNSNPPTFGGGTKVEIK

SEQ ID NO: 109 - protein, 106 aa, Artificial Sequence
  light chain
EIVLTQSPATLSLSPGERATLSCSVSSSVFYVHWYQQKPGQAPRLLIYDTSKLASGIPARFSGSGSGTDFTLTISSLEPEDFAVYYCQQWNSNPPTFGGGTKVEIK

SEQ ID NO: 110 - protein, 106 aa, Artificial Sequence
  light chain
EIVMTQSPATLSVSPGERATLSCSVSSSVFYVHWYQQKPGQAPRLLIYDTSKLASGIPARFSGSGSGTEYTLTISSLQSEDFAVYYCQQWNSNPPTFGGGTKVEIK

SEQ ID NO: 111 - protein, 106 aa, Artificial Sequence
  light chain
DIQMTQSPSSLSASVGDRVTITCSVSSSVFYVHWYQQKPGKAPKLLIYDTSKLASGVPSRFSGSGSGTDFTFTISSLQPEDIATYYCQQWNSNPPTFGGGTKVEIK

SEQ ID NO: 112 - protein, 106 aa, Artificial Sequence
  light chain
DIQMTQSPSSLSASVGDRVTITCSVSSSVFYVHWYQQKPGKAPKLLIYDTSKLASGVPSRFSGSGSGTDYTFTISSLQPEDIATYYCQQWNSNPPTFGGGTKVEIK

SEQ ID NO: 113 - protein, 106 aa, Artificial Sequence
  light chain
EIVMTQSPATLSLSPGERATLSCRASSSVFYLSWYQQKPGQAPRLWIYDTSKLASGIPARFSGSGSGTDFTLTISSLQPEDFAVYYCQQWNSNPPTFGGGTKVEIK

SEQ ID NO: 114 - protein, 11 aa, Artificial Sequence
  CDR1
RASQDISNYLN

SEQ ID NO: 115 - protein, 7 aa, Artificial Sequence
  CDR2
YTSRLHS

SEQ ID NO: 116 - protein, 9 aa, Artificial Sequence
  CDR3
QQGNTLPWT